(19) United States
(12) Patent Application Publication
(10) Pub. No.: US 2022/0130500 A1
(43) Pub. Date: Apr. 28, 2022

(54) RANDOMIZATION HONORING METHODS TO ASSESS THE SIGNIFICANCE OF INTERVENTIONS ON OUTCOMES IN DISORDERS

(71) Applicants: TONIX PHARMACEUTICALS HOLDING CORP., Chatham, NJ (US); TONIX PHARMACEUTICALS INC., Chatham, NJ (US)

(72) Inventors: Seth Lederman, South Dartmouth, MA (US); Philip B. Stark, Berkeley, CA (US); Ben Vaughn, Chapel Hill, NC (US)

(21) Appl. No.: 17/508,182

(22) Filed: Oct. 22, 2021

Related U.S. Application Data
(60) Provisional application No. 63/104,472, filed on Oct. 22, 2020.

(51) Int. Cl. G16H 10/20; G06F 17/18

(52) U.S. Cl. CPC G16H 10/20; G06F 17/18

(57) ABSTRACT

Systems and methods for an analysis of clinical trial data using randomization tests that honors the randomized design of the clinical trial are provided herein. In particular, a non-parametric analyzer implementing randomization tests and associated methods for analyzing clinical data is provided. The non-parametric analyzer receives clinical trial data comprising a data structure containing data corresponding to subjects in the clinical trial, where the subjects have been organized into treatment groups, including at least one control group. The non-parametric analyzer generates multiple treatment allocations of the data structure by reorganizing, at random, the subjects along with corresponding data to generate further groups. In some embodiments, the non-parametric analyzer determines the statistical significance based on an overall probability and the multiple allocations of the data structure. The overall probability may be generated via a combination analysis for comparing test statistics between groups of the data structure and the multiple allocations.

### CROSS-REFERENCE TO RELATED APPLICATIONS

This application claims priority and benefit from U.S. Provisional Application No. 63/104,472, filed Oct. 22, 2020, the contents of which is hereby incorporated by reference in its entirety.

### TECHNICAL FIELD OF THE DISCLOSURE

The present disclosure is directed to clinical data analysis that honors the actual randomized design of the experiments, and more particularly, to systems and methods for analyzing clinical data from randomized trials including those focused on treating psychiatric, sleep, pain, and neurological disorders.

### INTRODUCTION

Clinical trial data under randomized, double-blind studies are typically required by regulatory agencies, such as the United States Food and Drug Administration (FDA), to support marketing authorization of various therapeutic products (e.g., pharmaceutical or biological products). As used herein, the term common estimate-based p-value (“CEB p-value” hereinafter) refers to a nominal p-value that is calculated using typical assumptions that are inconsistent with the randomized design of the clinical trial. In particular, CEB p-values are computed using assumptions that may have little connection to the random assignment of subjects to treatments. Instead of testing the null hypothesis (i.e., treatment has no effect), the null hypothesis involves parameters of distributions and assumptions such as subjects' responses being a random sample from a specific distribution. The probability in such calculations does not come from randomized design of the experiment (i.e., the allocation of subjects to treatments). As used herein, the term “allocate” and its related forms may be referred to interchangeably with the term “allocate at random”. As used herein, the term “treatment allocation” refers to allocation, at random, of a subject to a treatment, treatment level, and/or treatment group including data corresponding to the subject. Instead, the probability arises from imposed assumptions including that the subjects were selected at random from larger hypothetical parametric populations. In particular, regulatory agencies often accept CEB p-values to support or reject marketing authorization at the time of the present disclosure. CEB p-values particularly refer to two-sided p-values (i.e., one-sided p-values multiplied by a factor of two when the one-sided p-values are in a direction favoring the active drug). For example, under current FDA guidelines, CEB p-values may be used to assess the separation of drug and placebo (or active comparator) on efficacy endpoints. Some regulatory agencies may also require positive support from more than one independent study for marketing authorization. For example, under current FDA guidelines, positive support entails at least two independent studies resulting in a CEB p-value (i.e., two-sided p-value) less than 0.05 (i.e., statistically significant under the current FDA guidelines). CEB p-values from one clinical trial are typically used to motivate and design subsequent clinical trials, but CEB p-values may lead to misleading predictions about the anticipated results of those subsequent studies and the efficacy of treatment, in particular when going from phase II to phase III trials. Many companies developing therapeutic products may have abandoned development of, e.g., potentially beneficial psychiatric drugs because CEB p-values failed to meet statistical significance, when in truth the drugs were truly beneficial or because interpretations of CEB p-values led to misleading predictions about the results of subsequent studies. CEB p-values may be misleading because the clinical trial analyses fail to honor the randomized design of the clinical trial, and/or use methods that impose unwarranted and invalid assumptions as part of standard practice. The use of CEB p-values may also have contributed to the lack of new drug development and to subsequent prolongation and expansion of major health problems from inadequately treated psychiatric conditions such as depression, post-traumatic stress disorder, schizophrenia and bipolar disorder.

Current standard practice in clinical and medical research employs randomization protocols that typically stratify by factors such as sex, clinical study site, and other characteristics to balance the study groups. Analyses under the current standard practice often apply parametric statistical tests to assess the null hypothesis (i.e., that the treatment has no effect) as a test of efficacy of the treatment, for example, in double-blinded trials. However, the standard parametric approach to assess the null hypothesis requires distributional assumptions that are often inconsistent with the current experimental practice and measurement process. In particular, CEB p-values are usually calculated under these inconsistent distributional assumptions. Consequently, the standard parametric approach (e.g., based on results from a parametric t-test, ANOVA, regression, or other method that treats the data as if they were a random sample from a parametric distribution) may sometimes produce misleading results due to the inapt distributional assumptions. For example, the Student's t-test assumes that the responses of the treatment and control groups are independent samples from normally distributed populations with equal means and equal variances (i.e., the independence and parametric assumptions). However, under common randomized designs of clinical trials, the treatment and control groups are not independent random samples from larger populations. Instead, a single population of subjects that meet the screening conditions for the trial is allocated at random into groups using a selected randomization protocol that makes the groups dependent (i.e., if an individual is in one group, that individual cannot be in any other group). Thus, the independence assumption that underlies many CEB p-values is not valid. Moreover, subjects' responses generally do not follow a hypothesized parametric distribution such as the normal distribution, even within groups. The hypothesized parametric distribution used to calculate the CEB p-value is not the probability distribution that comes from the experiment. Thus, parametric assumptions that underlie many CEB p-values based on parametric statistics may not be valid. As a result, a method based on independence and/or parametric assumptions may reject the null hypothesis even though the experiment as designed does not support rejecting the null hypothesis (e.g., the treatment actually is not effective) because the independence assumption is inconsistent with the randomized design and/or the parametric assumptions are inconsistent with the actual distribution. Conversely, a method that relies on independence and/or parametric assumptions may fail to reject the null hypothesis even though the experiment as designed supports rejecting the null hypothesis (e.g. the treatment is actually effective) because the independence and/or parametric assumptions about the clinical trial data are inconsistent with the randomization design and the nature of the measurements. In short, applying the standard parametric approach to the data from a randomized study fails to honor the randomized design for the experiment and adds distributional assumptions that have no substantive justification. Parametric tests in such randomized studies generally translate the scientific null hypothesis (i.e. the treatment is ineffective) into a quite different statistical hypothesis: the value of a parameter in a particular parametric model that is essentially unrelated to the experiment is equal to zero. The parametric methods generally answer the wrong question. For instance, Student's t-test answers the question: if the responses of the control group and the responses of the treatment group were independent random samples from two normally distributed populations with the same population mean and the same population variance, would it be unlikely to observe a difference in mean responses as large or larger than was observed? But in randomized trials, the responses of the control group and the treatment group are not independent random samples from normally distributed populations, so answering that question does not shed light on whether the treatment is effective or not. Therefore, in interpreting the data from randomized trials, there is a need for methods to assess the null hypothesis (i.e. that the treatment is not effective) while, at the same time, honoring the randomized design of clinical trials and the nature of the measurements.

### SUMMARY OF THE DISCLOSURE

In some embodiments, the present disclosure is directed to the statistical analysis of clinical trial data using randomization tests that, in some preferred embodiments, honor the randomized design of experiments and the non-parametric nature of the data. In particular, systems and methods are provided for a non-parametric analysis that implements a non-parametric combination of tests (NPCOT) to conduct a rigorous and robust analysis of medical and clinical research experiments and the data obtained from them. As used herein, the term “randomization tests” refers to univariate tests, multivariate tests, and including but not limited to NPCOT to build tests from multivariate observations.

In various embodiments of the disclosure, randomization tests are designed to honor the randomization of subjects into groups as it is conducted in practice with no additional assumptions aside from non-interference of subjects or participants. Non-interference refers to the assumption that a subject's response depends only on the treatment assigned to that subject and not on the treatments assigned to other subjects. As used herein, the term “statistical p-value” refers to a p-value that is determined without reliance on assumptions inconsistent with the randomized design as described earlier in the present disclosure, but rather, a p-value that relies only on the actual randomization performed and the observed data. In particular, a statistical p-value is not determined based on distributional assumptions but is instead determined based on the probability that arises from the randomized allocation of treatment to each subject. For example, determining a statistical p-value does not require assuming that subject's responses are a random sample from a larger hypothetical population or that the responses follow a normal distribution. Randomization tests respect the manner in which clinical trial participants are randomized into different treatment groups (e.g., drug and placebo groups). In practice, the subjects comprise a single population defined in part by availability and in part by inclusion and exclusion criteria (e.g., to reduce demographic bias). The inclusion and exclusion criteria also ensure that the subjects have the condition or conditions that the treatment under study is intended to address. That population may be randomized into multiple groups while approximately balancing the characteristics of the groups (e.g., treatment and control groups at individual sites and to balance ratio of sexes, etc. between groups, temporal sequence of enrollment and study period to balance external factors such as seasonal weather or pandemics). Randomization tests account for the dependence among the multiple groups properly and fully. Thus, all the probability assertions flow from the randomization protocol used in the experiment or trial. These attributes of randomization tests improve the reliability of the statistical inferences. In particular, randomization tests address a common problem for psychiatric, neurological, and subjective clinical trials based on multi-item assessment scales, where the results (e.g., CEB p-values) of trials by current methods (i.e., logistical and/or parametric) can be sometimes misleading, as discussed above, as to the actual effectiveness of the tested treatment. Accordingly, the sometimes misleading results may increase the risk of subsequent trials failing and mistakenly abandoning development of (e.g., potentially effective psychiatric) treatments, as well as the risk that ineffective treatments will be mistakenly believed to be effective and brought to market.

In some embodiments, the non-parametric analysis system implementing randomization tests of this disclosure is appropriate for analyzing clinical trials, including clinical trials that may contain measurement variables (e.g., scores and metrics) on discrete, bounded, and ordinal scales. In some embodiments, the clinical trial data include data for one or more assessment scales. For example, the data structure may include response data from a plurality of assessment scales. The assessment scales may include one or more assessment domains (e.g., categories or dimensions) and each assessment domain may include a plurality of assessment items (e.g., questions within the categories). Randomization tests may be used to assess the clinical trial data including the data based on one or more assessment scales. In some embodiments, more than one scale can be evaluated at the same time, in closed sequential analysis. As used herein, “closed sequential analysis” refers to an analysis involving statistical assessment in a designated sequence of trial or trial endpoints, in particular at a designated primary trial or trial endpoint and one or more designated secondary trials or trial endpoints in a specific order. For example, if the data are statistically significant on the primary endpoint, then secondary endpoints may be tested in a specific order. For example, if the data are not statistically significant at the primary endpoint, then secondary endpoints may be considered nominally positive but may not be used to show statistical significance. Examples of assessment scales and/or tests include the Clinician Administered PTSD Scale (e.g., CAPS-5), Alcohol Use Disorders Identification Test, Bergen Shopping Addiction Scale, ADHD Rating Scale, Disruptive Behavior Disorders Rating Scale, ALS Functional Rating Scale, Disability Rating Scale, International Cooperative Ataxia Rating Scale, Bush-Francis Catatonia Rating Scale, Behavior Rating Inventory of Executive Function, Rancho Los Amigos Scale, Paratonia scale, International Cooperative Ataxia Rating Scale, The Berg Balance Scale, Montreal Cognitive Assessment, Glasgow Outcome Scale, Toronto Western Spasmodic Torticollis Rating Scale, ALS Functional Rating Scale—Revised, multiple sclerosis functional composite, Stanford-Binet Intelligence Scales, Clinical Global Impression (CGI), Patient Global Impression (PGI), Quality of Life (QoL), Neuropsychological Impairment Scale, The Wechsler Scales, Engel Epilepsy Surgery Outcome Scale, Ballard Maturational Assessment, Yale Global Tic Severity Scale, Tourette Syndrome Clinical Global Impression and Shapiro TS Severity Scale, and hyperintensities scales.

In some embodiments, randomization tests honor the randomized design of the trial by excluding assumptions that are inconsistent with the randomized design (e.g., various distributional assumptions such as the independence and/or parametric assumptions). Randomization tests allow analysts to choose a suitable metric to gauge improvement that is meaningful clinically and statistically. For example, in some embodiments, a median and/or another quantile may be more appropriate summaries of scores and changes in scores on discrete, bounded, and ordinal scales than a mean because a mean implicitly assumes that the measurement scale is linear, while medians and percentiles make sense for any ordinal scales. Further, in some embodiments, randomization tests enable the analysis of multivariate data (e.g., response data from different categories from each subject). In such embodiments, the data structure may include multiple dimensions that correspond to different variables in the multivariate data. The multivariate data may include an arbitrary correlation structure (e.g., correlation among some or all variables). In some embodiments, randomization tests enable multivariate analysis while properly including such correlations. For example, each of the various individual CAPS items can be taken as a separate measurement that contributes evidence about the null hypothesis without losing information provided by the observed changes in the individual scores. For at least the above reasons, randomization tests are particularly useful, in contrast to the standard parametric approach, which lacks foundation under the present randomized design of experiments beyond familiarity and custom. In addition, in some embodiments, the randomization tests include a robust procedure to address missing data.

As described in the present disclosure, the non-parametric analysis system provides a rigorous, robust, and effective method to test hypotheses about many common, practical issues in clinical trials. The non-parametric analysis system may be useful, for example, to support marketing authorization for pharmaceutical, diagnostic and service products. Some non-limiting examples of such products include medications (e.g., antidepressants and antipsychotics), medical devices, and non-drug-based treatments (e.g., psychotherapy and cognitive behavioral therapy). For example, a treatment for post-traumatic stress disorder (PTSD) may include both medication and regular consultation. Some other non-limiting examples whose clinical treatment can be usefully analyzed in embodiments of this disclosure include psychological conditions or syndromes, psychiatric disorders, and central nervous system (CNS) diseases.

In some embodiments, the present disclosure is directed to a non-parametric analysis system and associated methods for testing hypotheses using clinical trial data while, at the same time, honoring the randomized design of the clinical trial. The clinical trial data may include data from a trial of a potential agent to treat, for example, a psychological condition, a psychological syndrome, a psychiatric disorder, a central nervous system disease, or a combination thereof. For example, the potential agent may be thought to be useful for treating PTSD. As used in the present disclosure, the term “non-parametric analysis system” may be referred to interchangeably with the term “non-parametric analyzer”. In some embodiments, the non-parametric analysis system may include an allocation generator, an allocation tracker, and/or a combination analyzer as part of or coupled to processing circuitry. In some embodiments, any of the associated methods may be fully or partially computer implemented. For example, the non-parametric analysis system may generate multiple treatment allocations using the allocation generator via processing circuitry. In some embodiments, combination analysis may be executed by the combination analyzer using processing circuitry.

In some embodiments, treatment allocations may be generated at random without constraints or criteria (e.g., based on a pseudo-random number generator). In some embodiments, treatment allocations may be generated with probabilities comparable to how the subjects from the clinical trial data were originally assigned to treatment groups (e.g., with constraints and/or balancing criteria similar to the original assignments). In some embodiments, treatment allocations are generated while maintaining group sizes comparable to the group sizes in the clinical trial data (e.g., using a randomization protocol without a stratifying factor). In some embodiments, treatment allocations are generated by executing an algorithm that is comparable to the algorithm used for assigning subjects to the original treatment groups. In some embodiments, treatment allocations are generated by executing the program (e.g., software) used for assigning subjects to the original treatment groups. Treatment allocations may be generated using any of the techniques or any combination of the techniques as described in the present disclosure.

In some embodiments, the non-parametric analysis system of this disclosure receives clinical trial data, including a data structure, and generates multiple versions of the data structure (e.g., multiple treatment allocations based on the data structure). In some embodiments, the data structure includes multivariate data. Randomization tests are designed to properly handle multivariate data (e.g., multi-item assessments). In some embodiments, generating multiple treatment allocations enables analyzing multivariate data while reducing or minimizing information loss. Components of the data structure may correspond to variables of the multivariate data. For example, a data structure may be a collection of vectors and/or scalars, each of which corresponds to a subject in the clinical trial, with each vector component comprising a measurement for a criterion of interest, in addition to a component that indicates the treatment received by the subject. In some embodiments, analyzing the multivariate data while reducing information loss includes determining importance weights for variables of the multivariate data. For example, a change in measured data for a first variable may be more pertinent to an effect from a treatment in the clinical trial than a change in measured data for a second variable. By reducing information loss, the importance of each variable (e.g., to the statistical significance) may be determined and/or assigned a corresponding statistical weight.

In some embodiments, the clinical trial data may be contained in a first data structure, where the first data structure is organized into a plurality of groups based on treatment levels. In some embodiments, the clinical trial data include data collected from trials at one or more study locations (i.e. sites) and the plurality of groups are further organized based on the one or more locations. One or more treatment levels may include, but are not limited to, a treatment intensity, a treatment dosage, and/or a treatment frequency. Outcomes on certain scales may be collected separately for the frequency and intensity of symptoms or syndrome manifestations. Such frequency and intensity assessments may be treated as separate variables within the clinical trial data. In some embodiments, when determining treatment level, biological markers and other information are evaluated simultaneously. In some embodiments, the data structure contains data corresponding to subjects in the clinical trial. The subjects may have been organized into a plurality of groups. In preferred embodiments, the subjects have been organized in groups based on assigned treatments or treatment levels in the clinical trial. In some embodiments, the clinical trial data, corresponding to each subject in each group of the plurality of groups, depend only on the treatment level of each group for which each subject is assigned. In some embodiments, the first data structure is organized prior to receiving the clinical trial data. This first data structure organized in the plurality of groups based on treatment levels may be referred to as the observed data having associated groups. In some embodiments, the non-parametric analysis system includes, as part of the randomization tests, organizing the observed data in the plurality of groups based on treatment levels. At least one group of the plurality of groups may be assigned a status as a control group. A control group, as used in the present disclosure, refers to a group undergoing a reference treatment or treatment level. As used in the present disclosure, a reference treatment refers to a treatment or any combination of treatments designated as a baseline to compare against results of the clinical trial. For example, if the treatment is directed to a new medication or active component, a control group may be given a placebo, instead of the new medication, to serve as a reference treatment level for the new medication or active component. In another embodiment, a control group is treated with a reference dosage of the new medication or active component to serve as a reference treatment level. In some embodiments, each control group has a corresponding reference treatment level depending on the criteria of interest. In some embodiments, the reference treatment levels may include any combination of treatment levels to serve as reference points for the clinical trial. For simplicity, the groups in a data structure that are organized based on treatment level, including any reference treatment level, may be referred to interchangeably as groups or treatment groups in the present disclosure. The treatment groups and control groups may be referred to collectively as groups or treatment groups.

In some embodiments, the clinical trial data include ordinal data, and generating multiple treatment allocations enables randomization tests to properly handle ordinal data when assessing the statistical significance, if any, of results from the clinical trial data and/or testing efficacy and safety of an agent, composition, treatment, or combination based on the clinical trial data. In such embodiments, the clinical trial data may include measurement data and/or categorical response data from subjects in the clinical trial for one or more criteria. In some embodiments, variables of the multivariate data may correspond to the multiple criteria. In some embodiments, components of the data structure may correspond to the multiple criteria. For example, the response data may include subject responses to a questionnaire with multiple questions, each question being, for example, directed to a separate criterion or a combination of criteria. Some exemplary questions include, but are not limited to, questions about: (a) a disorder or condition of a subject, (b) a state of a subject (including a physical, mental, or other state), (c) a presence of any symptoms, (d) a severity of one or more of the symptoms, and/or (e) any combination thereof. The response data may include responses to the questionnaire at multiple instances including before, during, and after the trial. For example, the data structure of the multivariate data may include baseline comparison data for multiple criteria corresponding to a subject in the clinical trial. As used herein, in some embodiments, the baseline comparison data is based on a comparison between data at a prior time point in or before the clinical trial and data at a different time point in or after the clinical trial. The baseline comparison data may include values on continuous scales, discrete scales, and/or categorical responses to multiple criteria.

In some embodiments, the non-parametric analyzer, as part of a randomization test, may include, selecting a test statistic or multiple test statistics for comparing the different groups prior to receiving the clinical trial data. Selecting the test statistics before the statistical analysis may avoid or reduce bias in the analysis of the clinical trial data. Some non-limiting examples of test statistics may include: (a) medians, (b) means, (c) least-squares means based on analysis of covariance (ANCOVA), (d) least-squares means based on mixed effect model repeated measures (MMRM), (e) percentiles, and (f) other measures of differences between distributions such as the Kolmogorov-Smirnov distance. For example, in the case of a treatment group and a control group, the test statistic may be the difference in the medians between the two groups. This test statistic may be sensible for ordinal data (e.g., on Likert scales) measured for each group. In some embodiments, when comparing least-square means based on MMRM, the MMRM may be similar to that used for a primary efficacy analysis in the clinical studies. In some embodiments, when comparing least-squares means based on ANCOVA, some non-limiting examples of covariates include covariates for baseline value, site, gender, ethnicity, and age.

In some embodiments, the non-parametric analysis system, as part of a randomization test, may execute one or more sensitivity analyses of the clinical trial data. In some embodiments, randomization tests may be used to determine whether a result based on the clinical trial data is due at least in part to a side effect of a treatment including when executing the sensitivity analysis. For example, the side effect may include a habit-forming effect such as an addictive or other reinforcing property due to the treatments. In some embodiments, randomization tests may be used to determine whether a result based on the clinical trial data is due at least in part to external factors, including unblinding the clinical trial including when executing the sensitivity analysis.

In some embodiments, generating the multiple treatment allocations enables analyzing the clinical trial data without reducing components of the data structure. In some embodiments, analyzing the clinical trial data without reducing components includes determining importance weights for the components of the data structure. For example, a first component of the data structure may be more pertinent to results from a treatment than a second component of the data structure. By not reducing the components, the importance of each component (e.g., to the statistical significance) may be determined and/or assigned a corresponding statistical weight. In some embodiments, generating the multiple treatment allocations includes generating a portion of treatment allocations of the clinical trial data that may represent potential treatment allocations of the clinical trial data. For example, a portion of treatment allocations may be generated according to a Monte Carlo method that may represent enough potential treatment allocations of the subjects. In some embodiments, the non-parametric analysis system, when generating the multiple treatment allocations, reorganizes the data structure into a further plurality of groups without regard to treatment level or status as a control group. In some embodiments, the non-parametric analysis system generates each of the treatment allocations for use in randomization tests. Additionally or alternatively, in some embodiments, the non-parametric analysis system generates and then stores the treatment allocations for use in randomization tests. For example, the processing circuitry may store the treatment allocations in memory as part of generating the allocations of subjects to treatments. A treatment allocation preferably is represented by a data structure of the same size as the first data structure but with subjects' data organized into different groups than the original clinical trial data.

In some embodiments, the multiple treatment allocations may be generated using a randomization protocol. In preferred embodiments, the randomization protocol is the same or similar protocol that generated the original groups for the clinical trial. In a non-limiting example, prior to starting a clinical trial, subjects may have been originally allocated into treatment groups using a first randomization protocol, where subjects have a first probability of being assigned to a treatment (i.e., assignment probability). For example, the assignment probability have originally been equally probable for each group based on attributes of the first randomization protocol. In this example, the first randomization protocol may have been designed to provide balanced groups (I) by allocating subjects in blocks of a particular block size (e.g., a block size of 8 subjects). The first randomization protocol may include (II) a set of parameters and (III) balancing criteria. The first randomization protocol may have been (IV) executed using a first computer program (e.g., proprietary software for randomizing subjects into groups). Attributes (I)-(IV) may influence assignment probability when using a randomization protocol. For this non-limiting example, in some embodiments, a different randomization protocol may include different attributes than (I)-(IV) from the first randomization protocol. Thus, the different randomization protocol may not have assignment probability that is comparable to the first randomization protocol. For this non-limiting example, in some embodiments, a similar randomization protocol may include one or more attributes (I)-(IV) from the first randomization protocol and different attributes. In particular, the similar randomization protocol may provide balanced groups with assignment probability comparable to the first randomization protocol (e.g., 52% to be in one out of two groups). For this non-limiting example, in some embodiments, a same randomization protocol may include most of the attributes (I)-(IV) (e.g., (I)-(III)). In particular, the same randomization protocol may provide balanced groups that have about the same assignment probability as the first randomization protocol (e.g., 50.1% to be in one out of two groups). The balanced groups may result in including different individual subjects.

In some embodiments, reorganizing the data structure into the further plurality of groups may include applying constraints (e.g., for reducing demographic bias) as part of a randomization protocol. In some embodiments, the reorganization includes executing a group algorithm following the randomization protocol including constraints to form balanced groups based on preselected criteria. Some non-limiting examples of preselected criteria include group size and demographic distribution (e.g., age, nationality, ethnicity and/or gender). For example, the first data structure may be organized based on grouping subjects prior to conducting the clinical trial using a first group algorithm following a first randomization protocol including a first criteria. For example, the group size may be preferably restricted to match sizes of the plurality of groups based on the treatment levels. The allocation generator (e.g., via processing circuitry) may execute the first group algorithm with criteria matching some or all of the first criteria to form different groups without regard to the respective treatments or the respective treatment levels in the clinical trial (e.g., to match the group size and/or demographic distribution).

In some embodiments, the non-parametric analysis system determines the statistical significance of the clinical trial results based on an overall probability and the multiple treatment allocations as part of a randomization test. Randomization tests may assess the statistical significance of clinical trial results based on the multiple treatment allocations without including distributional assumptions that characterize a parametric statistical analysis. In some embodiments, the non-parametric analysis system generates sufficiently many treatment allocations to achieve an expected precision, power, and/or statistical level of significance. In some embodiments, the non-parametric analysis system, when determining statistical significance of results based on clinical trial data and/or testing efficacy and safety of an agent, composition, treatment, or combination based on the clinical trial data, generates an overall probability based on the data structure and each of the multiple treatment allocations of the data structure. For example, the overall probability may include or be a statistical p-value.

In some embodiments, generating the overall probability based on the data structure and multiple treatment allocations preserves directionality of the overall probability. In such embodiments, preserving directionality may enable randomization tests to determine a statistical overall probability that includes or is a statistical p-value. The overall probability may be used to assess whether or not to reject a null hypothesis. For example, the overall probability may be a statistical p-value. Having a low statistical p-value may be sufficient evidence to reject a null hypothesis (e.g., depending on the regulatory agency). In some embodiments, the non-parametric analysis system, when generating the overall probability, executes a combination analysis as part of a randomization test to construct an overall test statistic from test statistics for individual dimensions of measurement and to determine an overall probability using the multiple treatment allocations. Constructing the overall test statistic may include comparing test statistics between different groups of the first data structure and/or may include comparing test statistics between different groups of the multiple treatment allocations. In some embodiments, the non-parametric analysis system executes the combination analysis more than once at different times. For example, randomization tests may include executing the combination analysis for data collected at different epochs and/or after different treatment durations to measure the changes in a given response over time. Some important times include but are not limited to: (a) before the trial, (b) at key milestones during the trial (e.g., at beginning, midpoint, and at end of the trial), and (c) at various times after the trial.

In some embodiments, the non-parametric analysis system of the disclosure, when executing the combination analysis to generate the overall probability, (i) determines test statistics for each component of the data structure and multiple treatment allocations for comparing between groups, (ii) determines empirical probabilities for each test statistic, (iii) combines the empirical probabilities, and (iv) generates the overall probability based on the combined empirical probabilities. In some embodiments, the non-parametric analysis system determines test statistics corresponding to components of the first data structure and multiple treatment allocations. For example, the test statistics may involve comparing medians for each component of the data structure and across the multiple treatment allocations. In some embodiments, the empirical probabilities are determined based on ranking the test statistics. In some embodiments, the non-parametric system analyzer of the disclosure, when combining the empirical probabilities, applies a combining function to the empirical probabilities. Some non-limiting examples of combining functions may include the Fisher combining function, the Liptak combining function, the Stouffer combining function, or some combination thereof. For example, the combining function may be the Fisher combining function, −2 Σᵢ₌₁^{N} ln(pᵢ). In some embodiments, randomization tests involve, prior to receiving clinical trial data, selecting a combining function.

In some embodiments, some data may be missing for one or more subjects in the clinical trial. For example, in a CAPS-5 study, a subject may not have answered particular questions, resulting in missing response data. The non-parametric analysis system of this disclosure tracks which groups contain the subject in each of the multiple treatment allocations. In some embodiments, the non-parametric analysis system tracks which groups contain the subject when generating the multiple treatment allocations. The non-parametric analysis system of the disclosure may then organize the combined empirical probabilities based on the tracking of the subject. For example, the non-parametric analysis system may categorize the combined empirical probabilities in correspondence with the groups containing the subject in each of the multiple treatment allocations. The non-parametric analysis system then calculates a test statistic for comparing the categorized combined empirical probabilities.

In some embodiments, the non-parametric analysis system, when calculating the test statistic, may apply a transformation to each of the categorized combined empirical probabilities. For example, the non-parametric analysis system may compare logarithms or other suitable forms of the categorized combined empirical probabilities. In some embodiments, the non-parametric analysis system, when calculating the test statistic, may calculate a Hodges-Lehmann-Sen estimator for comparison. In some embodiments, the test statistic measures a change in the overall probability due to the missing portion of the clinical trial data associated with the subject. In some embodiments, when analyzing survival data, the test statistic accounts for censoring, such as left censoring, interval censoring, or right censoring. In some embodiments, the non-parametric analysis system, when tracking which groups contain the subject, generates a mapping of the subjects comprising each groups for each of the multiple treatment allocations. Alternatively, in some embodiments, the non-parametric analysis system of the disclosure, when tracking which groups contain each subject, stores identifiers of the groups containing each subject for each of the multiple treatment allocations.

In some embodiments, the non-parametric analysis system may rank a subject based on the change in one or more test statistics due to the missing portion of the clinical trial data associated with the subject. Additionally or alternatively, the non-parametric analysis system may impute the missing clinical trial data associated with each subject based on the tracking of the subject. For example, by tracking which groups contain the subject, the effect due to missing clinical trial data associated with the subject may be determined for each group and the missing clinical trial data may be imputed. In some embodiments, the missing data may be imputed based on tracking the subject more than once at different times before, during, or after the trial.

In some embodiments, randomization tests may be used to evaluate results of clinical trials to determine aspects of the design of one or more future clinical trials. For example, randomization tests may be used to determine how the size of a first clinical trial should be reduced or increased in a future clinical trial. In some embodiments, randomization tests may be used to evaluate interim results of a clinical trial for re-setting and/or adapting group sizes in adaptive clinical trials. For example, in some embodiments, randomization tests may be used during a clinical trial to assess the group sizes and/or effect of treatment regimens due to changing circumstances in an adaptive clinical trial. Accordingly, the size and/or number of groups or treatment levels may be adjusted during the on-going trial. Example techniques for a-spending (e.g., with a 1st stage of α=0.005) include but are not limited to: (i) the design method, (ii) Wang & Tsiatis method, (iii) Pocock Design method, (iv) O'Brien & Fleming method and (v) Lan & DeMets method.

In some embodiments, randomization tests may be used to evaluate safety of one or more treatments studied in clinical trials. For example, randomization tests may be used to assess whether toxicity, morbidity, or other undesirable outcomes potentially observed in a subject in the clinical trial are due to the treatment. For example, an inapt statistical assessment (e.g., based on distributional assumptions) may have been used for improper support (e.g., to establish an alleged toxicity based on invalid p-values). Randomization tests as described in the present disclosure may be used to show proper evidence in support of a different result (e.g., an alleged toxicity is not present or too minor to be have detrimental effects).

### BRIEF DESCRIPTION OF THE DRAWINGS

The present disclosure, in accordance with one or more various embodiments, is described with reference to the following figures. The drawings are provided for purposes of illustration only and merely depict example embodiments. The drawings are provided to facilitate an understanding of the concepts disclosed herein and do not limit the breadth, scope, or applicability of these concepts in any way. It should also be noted for clarity and ease of illustration that the drawings are not necessarily made to scale.

FIG. 1 shows an illustrative example of a system analyzing clinical trial data, in accordance with some embodiments of the present disclosure;

FIG. 2 shows an illustrative block diagram of a system analyzing clinical trial data, in accordance with some embodiments of the present disclosure;

FIG. 3 shows an illustrative block diagram of a system for analyzing clinical trial data, in accordance with some embodiments of the present disclosure;

FIG. 4 shows an illustrative block diagram of a system generating treatment allocations of clinical trial subjects, in accordance with some embodiments of the present disclosure;

FIG. 5 shows illustrative examples of treatment allocations of clinical trial subjects, in accordance with some embodiments of the present disclosure;

FIG. 6 shows an illustrative example of a data structure containing clinical trial data that has missing portions of the data, in accordance with some embodiments of the present disclosure;

FIG. 7 shows illustrative examples of data structures containing treatment allocations of clinical trial data having missing portions of the data, in accordance with some embodiments of the present disclosure;

FIGS. 8A and 8B (referred to collectively as FIG. 8 hereinafter) each show a flow diagram of an illustrative process for analyzing clinical trial data, in accordance with some embodiments of the present disclosure;

FIGS. 9A and 9B (referred to collectively as FIG. 9 hereinafter) each shows a flow diagram of an illustrative process for generating treatment allocations of clinical trial subjects, in accordance with some embodiments of the present disclosure;

FIG. 10 shows a flow diagram of an illustrative process for generating overall probabilities, in accordance with some embodiments of the present disclosure;

FIG. 11 shows a flow diagram of an illustrative process for analyzing clinical trial data, in accordance with some embodiments of the present disclosure;

FIG. 12 shows a flow diagram of an illustrative process for accounting for or imputing missing clinical trial data associated with a subject, in accordance with some embodiments of the present disclosure;

FIG. 13 shows a flow diagram of an illustrative process for tracking groups containing a subject associated with the missing clinical trial data, in accordance with some embodiments of the present disclosure;

FIG. 14 shows example tables comparing illustrative metrics of a randomization test, in accordance with some embodiments of the present disclosure;

FIG. 15 shows example tables comparing illustrative metrics of a randomization test, in accordance with some embodiments of the present disclosure;

FIGS. 16-24 show illustrative stages that may be part of randomization tests, in accordance with some embodiments of the present disclosure;

FIGS. 25-26 show example tables comparing illustrative metrics of a randomization test without and with the same randomization protocol from originally assigning subjects to treatment groups, in accordance with some embodiments of the present disclosure;

FIG. 27 shows a flow diagram of an illustrative process for determining statistical significance of results based on clinical trial data, in accordance with some embodiments of the present disclosure; and

FIG. 28 shows example tables comparing illustrative metrics of a randomization-based test combined with multiple imputation, in accordance with some embodiments of the present disclosure.

### DETAILED DESCRIPTION OF THE DISCLOSURE

### General Techniques and Definitions

Unless otherwise defined herein, scientific and technical terms used in this application shall have the meanings that are commonly understood by those of ordinary skill in the art. In case of conflict, the present specification, including definitions, will control.

The practice of the present disclosure will employ, unless otherwise indicated, suitable techniques of non-parametric statistics and data analysis in medical and clinical research, which techniques are within the skill of the art. Such techniques are explained in more detail in the literature including: Pesarin F, Salmaso L. Permutation tests for complex data. Theory, applications and software. Chichester: John Wiley & Sons, Ltd. (2010): Chapter 4, “The Nonparametric Combination Methodology” pp. 117-175; Arboretti, R. et al. “Test statistics in medical research: traditional methods vs multivariate NPC permutation tests.” Urology 2015; 85 (2): 130-136. DOI: 10.5301/uro.5000117; Rosenberger, W F, Uschner, D, Wang, Y. Randomization: The forgotten component of the randomized clinical trial. Statistics in Medicine. 2019; 38: 1-12. https://doi.org/10.1002/sim.7901. Each of the aforementioned publications is hereby expressly incorporated herein by reference in their respective entireties.

The term “including” is used to mean “including, but not limited to.” “Including” and “including but not limited to” are used interchangeably.

Any example(s) following the term “e.g.” or “for example” are not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. As used herein, the term “about” modifying the quantity of an parameter, calculation, or measurement described in the disclosure or employed in the methods of the disclosure refers to variation in the numerical quantity that can occur, for example, through typical measuring and/or handling procedures used for statistical assessments; through inadvertent error in these procedures. The term “about” also encompasses amounts that differ due to different conditions in medical and clinical experiments. Whether or not modified by the term “about”, the paragraphs include equivalents to the quantities. Reference to “about” a value or parameter herein also includes (and describes) embodiments that are directed to that value or parameter per se. For example, description referring to “about X” includes the description of “X.” Numeric ranges are inclusive of the numbers defining the range.

### Definitions

The following terms, unless otherwise indicated, shall be understood to have the following meanings:

The terms “patient,” “subject,” “participant,” and “individual” are used interchangeably herein and refer to either a human or a non-human animal. These terms include mammals, such as humans, primates, livestock animals (including bovines, porcines, camels, etc.), companion animals (e.g., canines, felines, etc.), zoo animals and rodents (e.g., mice and rats), and other animals used in research (e.g., rabbits).

The term “treatment” refers to any form of therapy or combination of therapies used in attempting to remediate a health problem, particularly stemming from a condition, a syndrome, a disorder, or a disease.

The term “control group” refers to a group undergoing a reference treatment.

The term “reference treatment” refers to a treatment or any combination of treatments designated as a baseline against which to compare or evaluate a studied treatment.

The term “multivariate” and “multidimensional” may be used interchangeably herein and refer to having multiple variables. The term “multivariate data” refers to a set of measurements for multiple variables for a collection of individuals.

The term “random” as used in the present disclosure refers to events occurring as if by chance while subject to particular constraints and/or criteria unless specified otherwise.

The term “treatment allocation” refers to assignation, at random, of a subject to a treatment, treatment level, and/or treatment group including data corresponding to the subject.

The term “randomization tests” refers to univariate tests, multivariate tests, and including but not limited to NPCOT to build tests from multivariate observations.

The term “closed sequential analysis” refers to an analysis involving statistical assessment in a designated sequence of trials or trial endpoints, in particular, at a designated primary trial or trial endpoint and at one or more secondary trials or trial endpoints in a specific order.

The term “test statistic” refers to a quantity derived from a sample (i.e., a statistic) used in statistical testing (e.g., null hypothesis testing).

The term “p-value” refers to the observed value of a random variable (X) that, under the assumption that a null hypothesis of interest is true, has probability (Pr) not exceeding a value (x) of not exceeding x, for every x between zero and one. That is, a p-value is the observed value of a random variable X for which, if the null hypothesis is true, Pr(X≤x)≤x for all x between 0 and 1.

The term “common estimate-based p-value” or “CEB p-value” refers to a nominal p-value that is calculated on the basis of typical assumptions, which typically are inconsistent with the randomized design of the clinical trial.

The term “statistical p-value” refers to a p-value that is determined without including assumptions inconsistent with the randomized design of the clinical trial. That is, a statistical p-value determines Pr(X≤x) based on the probability that arises from the randomized allocation of treatment.

Each embodiment described herein may be used individually or in combination with any other embodiment described herein.

### Overview

The present disclosure, in its various embodiments, is directed to clinical data analyses and systems honoring the randomized design of experiments, and more particularly, to non-parametric analyses, systems, and associated methods, including randomization tests for analyzing clinical data from treatment trials and research experiments including, in some embodiments, trials and experiments focused on attempting to treat psychiatric, sleep, pain, and neurological disorders.

### Detailed Description of Various Embodiments of this Disclosure

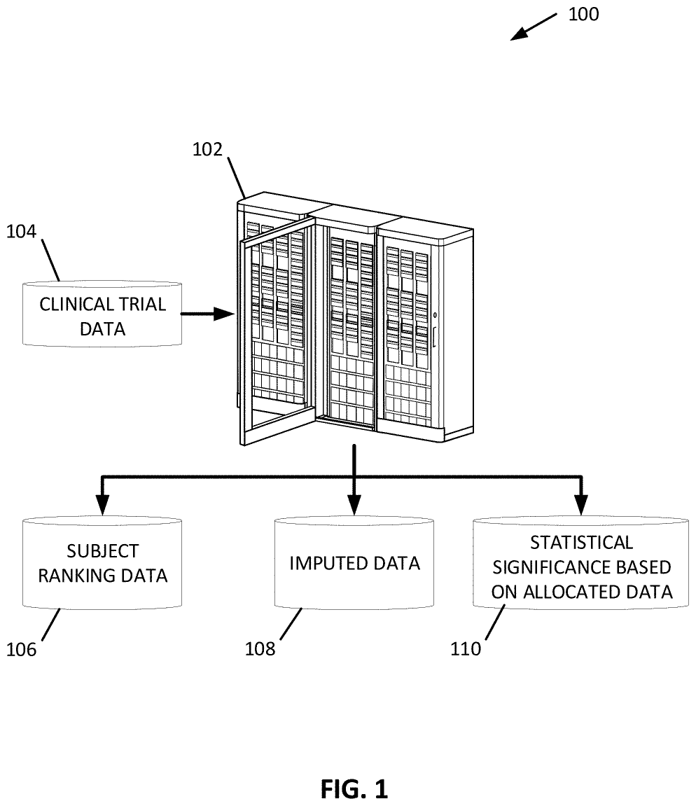

FIG. 1 shows an illustrative example of system 100 analyzing clinical trial data 104 non-parametrically, in accordance with some embodiments of the present disclosure. System 100 includes non-parametric analyzer 102, clinical trial data 104, and various output data 106-110. Non-parametric analyzer 102 includes an implementation of a randomization test. Clinical trial data 104 may be contained in a data structure, where the data structure is organized into a plurality of groups based on treatment levels. Clinical trial data 104 may include data from a trial of a potential agent to treat various conditions, diseases, and disorders, including a psychological condition, a psychological syndrome, a psychiatric disorder, a central nervous system disease, or a combination thereof. For example, the potential agent may be for treating PTSD. Non-parametric analyzer 102 receives clinical trial data 104. Non-parametric analyzer 102 may perform randomization tests in order to generate subject ranking data 106, imputed data 108, and/or statistical significance based on allocated data 110 (hereinafter referred to as statistical significance data for brevity). Subject ranking data 106 may be used to rank subjects based on effect of missing data associated with a respective subject. Imputed data 108 may be used to impute the missing data. Statistical significance data 110 may include overall probabilities (e.g., statistical p-values). For example, statistical significance data 110 may be used to assess a null hypothesis for clinical trial data 104.

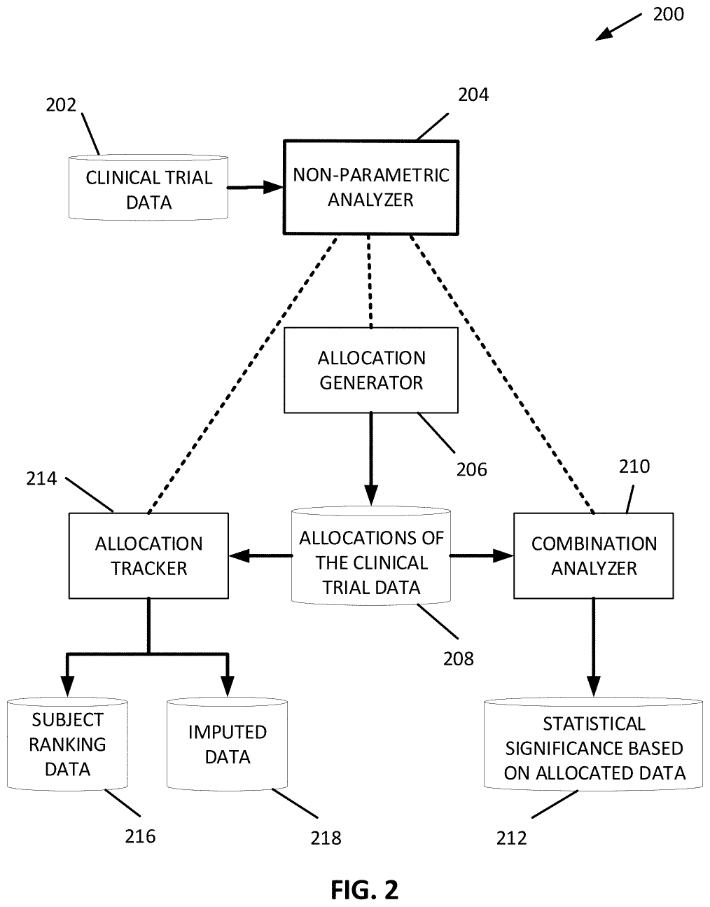

FIG. 2 shows an illustrative block diagram of system 200 analyzing clinical trial data 202 non-parametrically, in accordance with some embodiments of the present disclosure. In some embodiments, system 200 may include or be system 100. System 200 includes non-parametric analyzer 204. Non-parametric analyzer 204 includes or may be coupled to allocation generator 206, combination analyzer 210, and allocation tracker 214. In some embodiments, components 206, 210, and 214 may be part of or coupled to processing circuitry included in non-parametric analyzer 204. Non-parametric analyzer 204 generates allocations of data 208 using allocation generator 206. Allocations of data 208 contain treatment allocations of clinical trial data 202. Allocations of data 208 may include clinical trial data 202. Allocations of data 208 may be used in combination analyzer 210 and/or allocation tracker 214 as part of a randomization test. Combination analyzer 210 processes allocations of data 208 (e.g., via processing circuitry) and generates statistical significance data 212. As previously noted, statistical significance data 212 may be used to assess a null hypothesis based on clinical trial data 202. Allocation tracker 214 may track which groups of the treatment allocations contain one or more subjects using allocations of data 208, where some clinical trial data are missing for a subject or subjects. Allocation tracker 214 may generate subject ranking data 216 and imputed data 218 based on the tracking of subjects. Subject ranking data 216 may be used to rank subjects associated with missing data according to the effect of the associated missing data on the statistical significance (e.g., the change in the overall probabilities over the multiple allocations). As part of a randomization test, allocation tracker 214 may impute the missing data based on tracking the subject (e.g., in order to fill in the missing data).

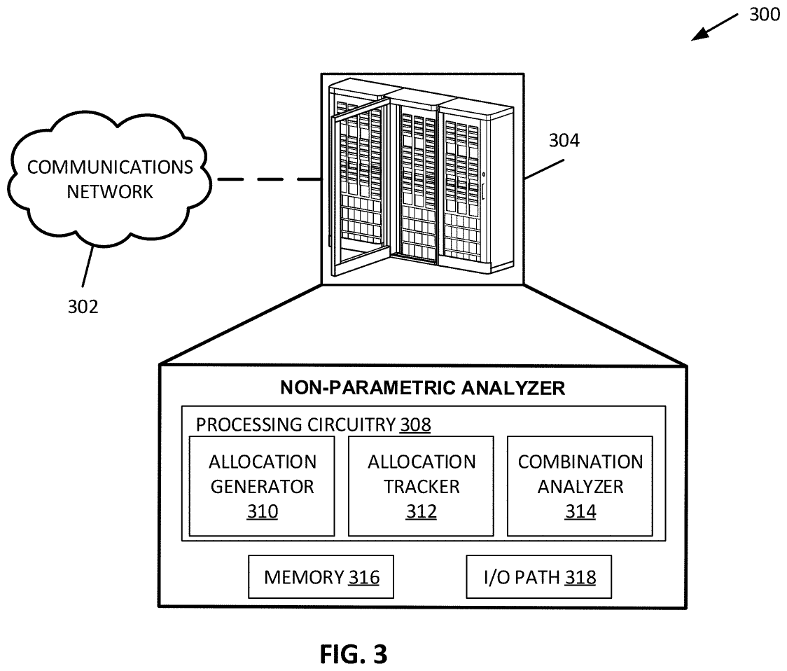

FIG. 3 shows an illustrative block diagram of system 300 for analyzing clinical trial data, in accordance with some embodiments of the present disclosure. System 100 and/or system 200 may, in some embodiments, include any or all of system 300. Although FIG. 3 shows system 300 as including a number and configuration of individual components, in some embodiments, any number of the components of system 300 may be combined and/or integrated as one device. System 300 includes non-parametric analyzer 304, where non-parametric analyzer 304 may be coupled to communications network 302 configured for receiving and transmitting data to a remote server. For example, non-parametric analyzer 304 may receive clinical trial data from communications network 302 and transmit any resulting data after executing a randomization test. Communications network 302 may include the Internet and/or any other suitable wired and/or wireless communications paths, networks and/or groups of networks. It should be noted that non-parametric analyzer 304 may be coupled to computing equipment via communications network 302.

Non-parametric analyzer 304 includes processing circuitry 308, memory 316, and input/output (I/O) path 318. Processing circuitry 308 includes allocation generator 310, allocation tracker 312, and combination analyzer 314. While system 300 is shown in one configuration, it should be noted that system 300 may be in any other suitable configuration. In some embodiments, system 300 is a remote server hosting an application (e.g., an implementation of randomization tests). In some embodiments, non-parametric analyzer 304 works in conjunction with a computing device coupled through communications network 302 to implement certain functionality described herein in a distributed or cooperative manner. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores). In some embodiments, processing circuitry may be distributed across multiple separate processors, for example, multiples of the same type of processors (e.g., two Intel Core i9 processors) or multiple different processors (e.g., an Intel Core i7 processor and an Intel Core i9 processor).

Memory 316 may be an electronic storage device. As referred to herein, the phrase “memory”, “electronic storage device”, or “storage device” should be understood to mean any device for storing electronic data, computer software, or firmware, such as random-access memory, read-only memory, hard drives, optical drives, solid state devices, quantum storage devices, or any other suitable fixed or removable storage devices, and/or any combination of the same. Memory 316 and/or storages of other components of system 300 may be used to store various types of data including metadata. Non-volatile memory may also be used (e.g., to launch a boot-up routine and other instructions). Cloud-based storage may be used to supplement memory 316 or instead of memory 316. In some embodiments, processing circuitry 308 executes instructions for an application (e.g., a randomization test) stored in memory (e.g., memory 316). Specifically, processing circuitry 308 may be instructed by the application to perform the functions discussed herein. In some embodiments, any action performed by processing circuitry 308 may be based on instructions received from the application. For example, the application may be implemented as software or a set of executable instructions that may be stored in memory 316 and executed by processing circuitry 308.

In client/server-based embodiments, processing circuitry 308 may include communications circuitry (e.g., I/O path 318) suitable for communicating with an application server (e.g., non-parametric analyzer 304) or other networks or servers. The instructions for carrying out the functionality described herein may be stored on the application server. Communications circuitry may include a cable modem, an Ethernet card, or a wireless modem for communication with other equipment, or any other suitable communications circuitry. Such communications may involve the Internet or any other suitable communications networks or paths (e.g., communications network 302).

Non-parametric analyzer 304 may transmit and receive content and data via one or more of I/O paths 318. I/O path 318 may be or include appropriate communications circuitry. For instance, I/O path 318 may include a communications port configured to transmit and/or receive clinical trial data and probability data via communications network 302. Processing circuitry 308 may be used to send and receive commands, requests, and other suitable data using I/O path 318.

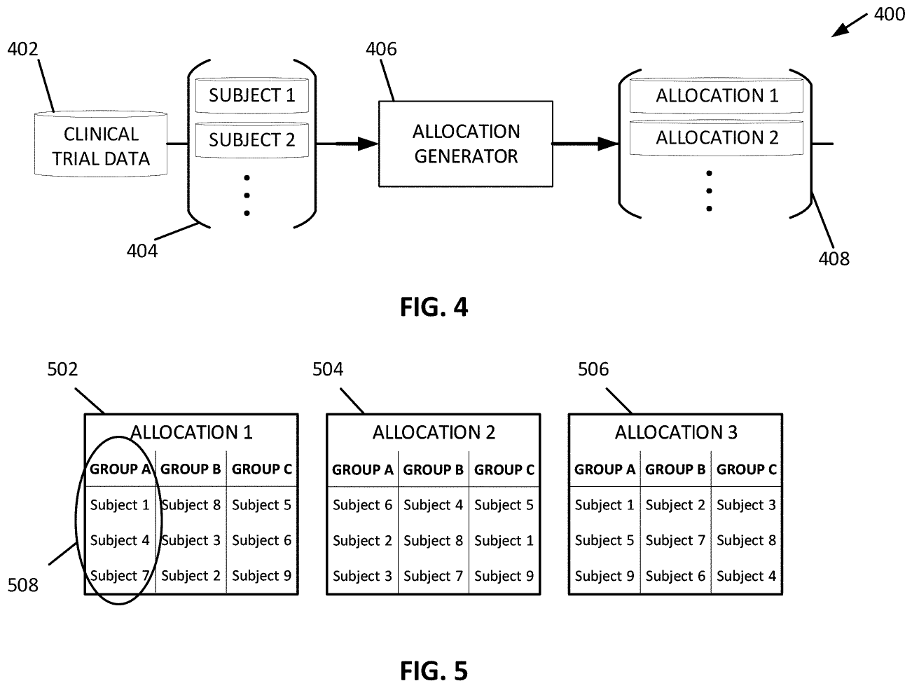

FIG. 4 shows an illustrative block diagram of system 400 generating allocations of clinical trial data 402, in accordance with some embodiments of the present disclosure. Clinical trial data 402 contain subject data 404 for multiple subjects in the trial. Subject data 404 may include a data structure with multivariate data for each subject in the clinical trial. Subject data 404 may be organized into groups (e.g., based on treatment levels). In some embodiments, variables of the multivariate data may correspond to multiple criteria. In some embodiments, components of the data structure may correspond to multiple criteria. Subject data 404 may include measurement data and/or categorical response data for each subject in the clinical trial for multiple criteria (e.g., Subject 1 response data). For example, the response data of subject data 404 may include subject responses to a questionnaire with multiple questions, each question for a separate criterion or a combination of criteria. Some exemplary questions include but are not limited to questions about: (a) a disorder or condition of a subject, (b) a state of a subject (including a physical, mental, or other state), (c) a presence of any symptoms, (d) a severity of one or more of the symptoms, and/or (e) any combination thereof.

Allocation generator 406 may generate, based on subject data 404, allocations of clinical trial data 402. For example, allocation generator 406 may reorganize the groups including, in a preferred embodiment, swapping subjects according to the randomization protocol applied in organizing the groups in clinical trial data 402. For example, allocation generator 406 may, at random, select subjects without regard to the organized groups to form a allocation containing different groupings of the subjects. Any suitable technique may be implemented in allocation generator 406 to generate allocations of data 408. Allocated data 408 contains the allocations of clinical trial data 402 and may include the clinical trial data 402.

FIG. 5 shows illustrative examples of allocations of clinical trial data, in accordance with some embodiments of the present disclosure. Data structures 502-506 each contain a allocation of clinical trial data (e.g., clinical trial data 402). Data structures 502-506 may be example allocations of clinical trial data (e.g., allocations of data 408). While the following refers to data structure 502, it should be noted that any allocation may be described similarly. Data structure 502 contains a first allocation of clinical trial data with three groups as shown. In the first allocation, Subjects 1-9, in conjunction with their corresponding trial data, are placed in Groups A-C in a different order than in the clinical trial data. For example, Group A at 508 contains Subjects 1, 4, and 7, along with corresponding trial data. Allocation generator 406 may generate as many allocations as desired for use in a randomization test. Preferably, allocations of data 408 does not include duplicates (e.g., to save storage space). However, randomization tests may allow for duplicate allocations. In some embodiments, allocation generator 406 may generate a portion of potential allocations of the clinical trial data. For example, a portion of possible allocations may be generated according to a Monte Carlo model that may represent multiple possible allocations of the clinical trial data.

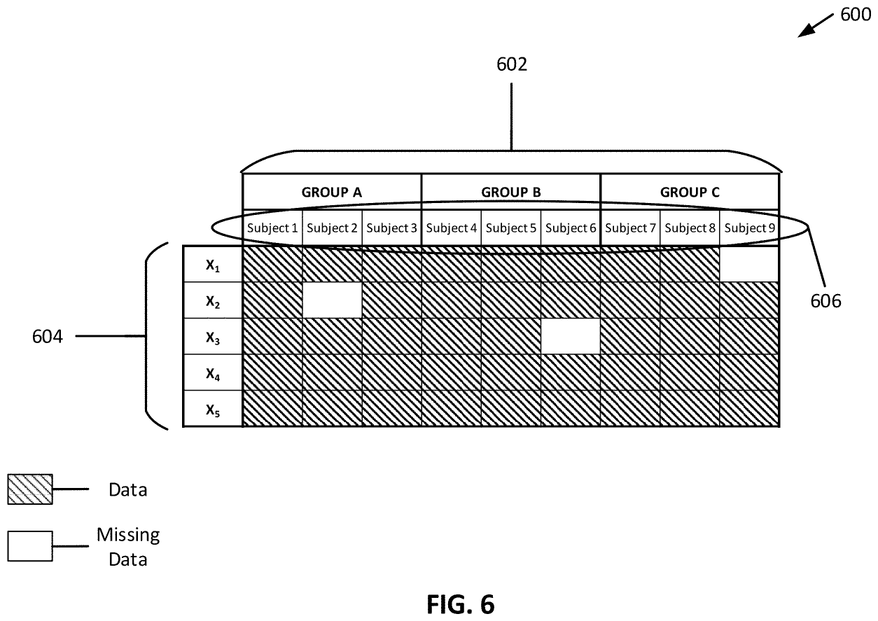

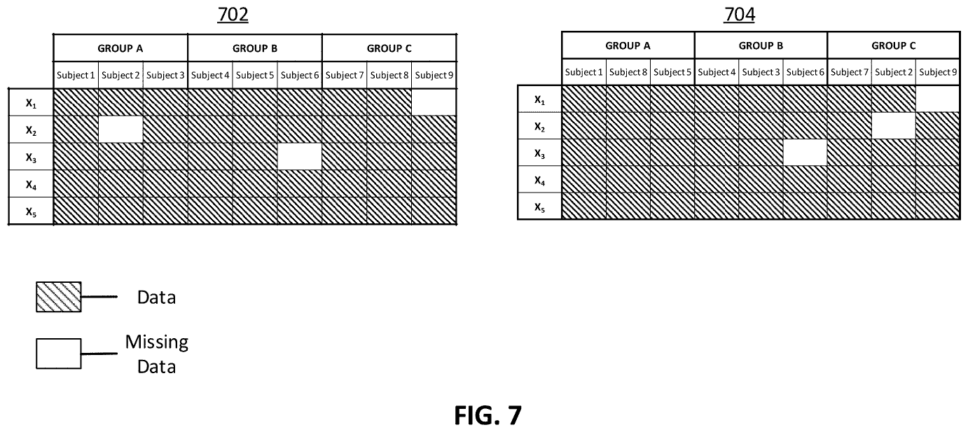

FIG. 6 shows an illustrative example of data structure 600 containing clinical trial data having missing portions of the data, in accordance with some embodiments of the present disclosure. FIG. 7 shows illustrative examples of allocations of clinical trial data having missing portions of the data, in accordance with some embodiments of the present disclosure. FIGS. 6 and 7 indicate filled data cells as shaded and blank cells as missing data. Data structure 600 includes groups 602 (e.g., Groups A-C), subject labels 606 (e.g., Subjects 1-9), and variables 604, (e.g. X₁-X₅). Groups 602 has the subjects evenly distributed between Groups A-C. It should be noted that, in some embodiments, the groups need not be the same size. Each subject has an associated column of data, which may be a vector of data as previously noted. Each cell in the column corresponds with a variable at 604. As shown in data structure 600, subjects 2, 6, and 9 have a missing portion of data. Referring to FIG. 7, data structure 702 is grouped in the same way as data structure 600. Data structure 704 shows an allocation of data structure 600.

Data structures 702 and 704 are shown side by side for comparison. In particular, subjects 2, 6, and 9 are in different groups at 704 than at 702. The missingness of subjects 2, 6, and 9 have moved in conjunction with their corresponding subjects and may affect any of the probabilities as generated in a randomization test using the allocations of data.

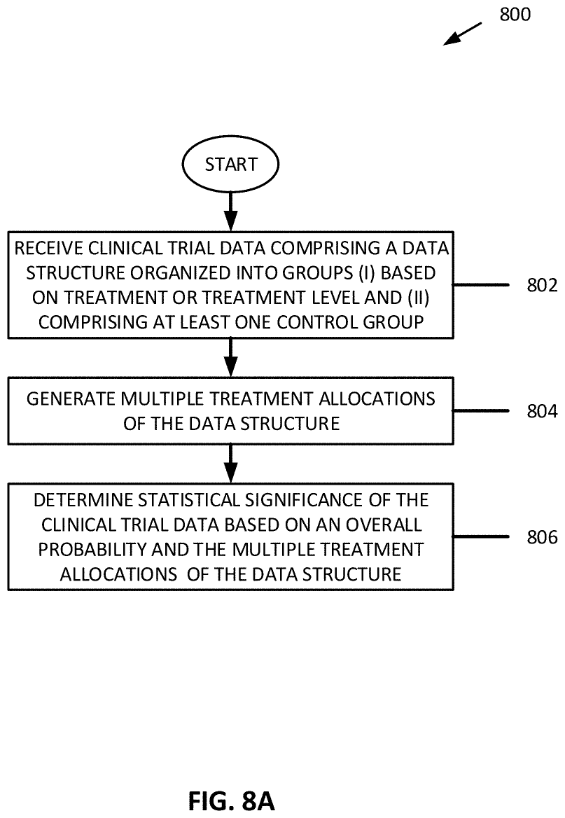

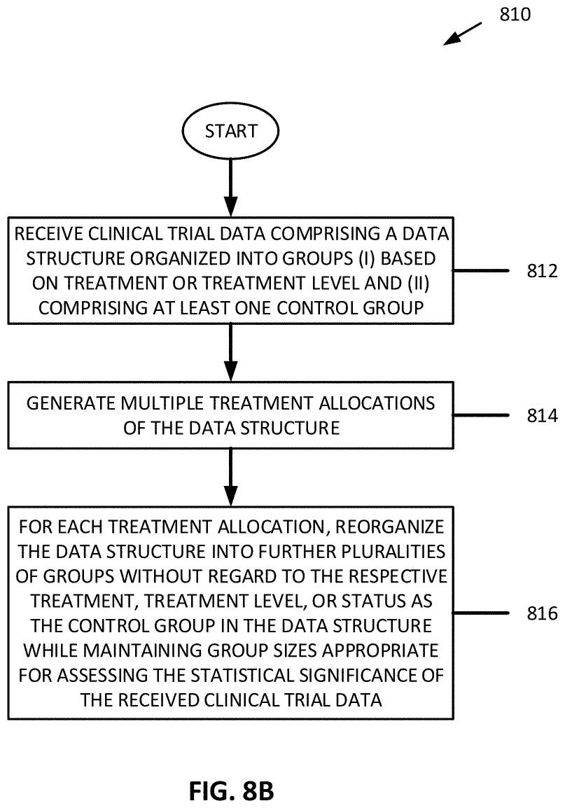

FIGS. 8A and 8B (i.e., FIG. 8) each show a flow diagram of an illustrative process for analyzing clinical trial data, in accordance with some embodiments of the present disclosure. FIG. 8A shows a flow diagram of an illustrative process 800 for analyzing clinical trial data. At 802, clinical trial data is received. The clinical trial data may include a data structure with multivariate data. As previously noted, the clinical trial data may be organized into a plurality of groups based on treatment levels and/or other criteria. One or more of the groups may be assigned as a control group. For example, one group may have been given a placebo in order for the group to act as a baseline reference for the treatment levels. At 804, multiple allocations are generated using the data structure. At 806, statistical significance is determined for the clinical trial data based on an overall probability and the multiple treatment allocations. FIG. 8B shows a flow diagram of an illustrative process 810 for analyzing clinical trial data. At 812, clinical trial data is received. The clinical trial data may include a data structure with multivariate data. As previously noted, the clinical trial data may be organized into a plurality of groups based on treatment levels and/or other criteria. One or more of the groups may be assigned as a control group. For example, one group may have been given a placebo in order for the group to act as a baseline reference for the treatment levels. At 814, multiple allocations are generated using the data structure. At 816, the data structure is reorganized for each allocation into further groups while maintaining group sizes appropriate for assessing statistical significance of results based on the clinical trial data and/or testing the efficacy and safety of an agent, composition, treatment, or combination based on the clinical trial data.

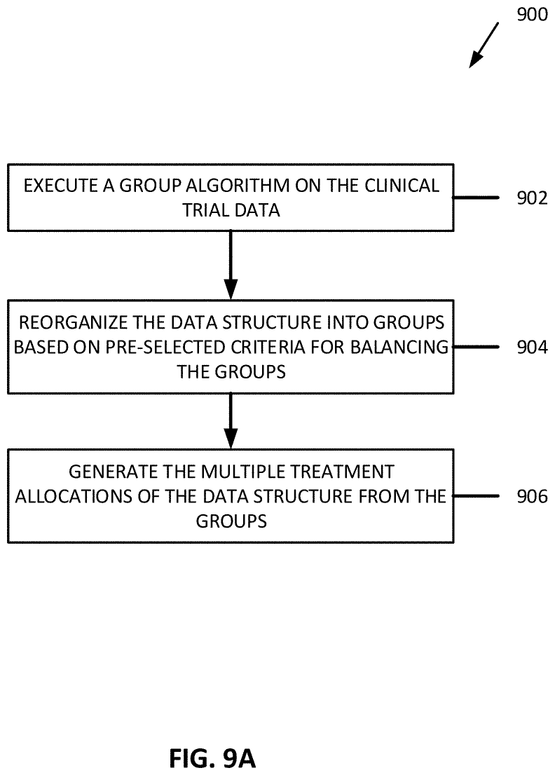

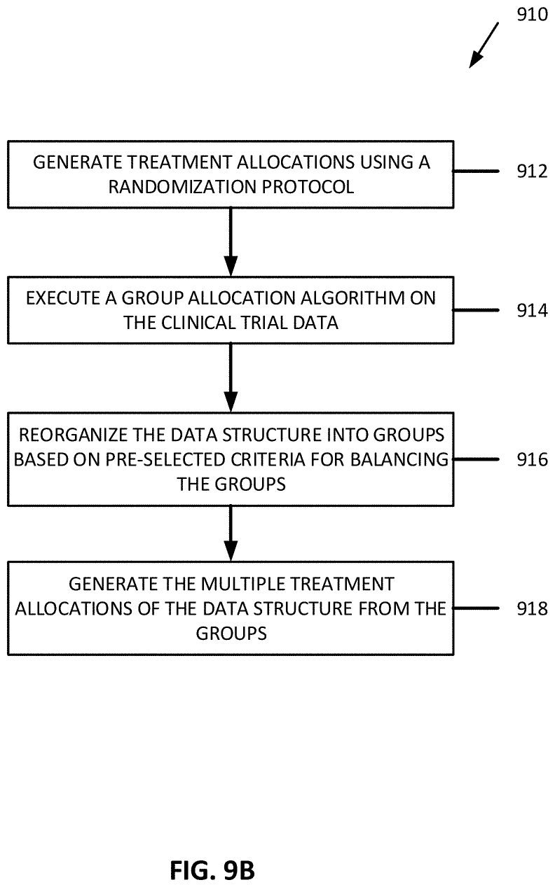

FIGS. 9A and 9B (referred to collectively as FIG. 9 hereinafter) each shows a flow diagram of an illustrative process for generating treatment allocations of clinical trial subjects, in accordance with some embodiments of the present disclosure. FIG. 9A shows a flow diagram of an illustrative process 900 for generating treatment allocations of clinical trial data. At 902, a group algorithm is executed on the clinical trial data. At 904, the data structure is reorganized into different groups than the clinical trial data (e.g., using the group algorithm). The reorganization, subject to preselected criteria, may be performed for balancing the groups. Preferably, the group algorithm that is used has one or more of the preselected criteria matched with the criteria used to organize the groups in the original clinical trial data. At 906, multiple treatment allocations are generated based on the reorganization of the groups. FIG. 9B shows a flow diagram of an illustrative process 910 for generating treatment allocations of clinical trial data. At 912, the treatment allocations are generated using a randomization protocol. At 914, a group algorithm is executed on the clinical trial data (e.g., using the randomization protocol). At 916, the data structure is reorganized into different groups than the clinical trial data. The reorganization, subject to preselected criteria, may be performed for balancing the groups. Preferably, the group algorithm that is used has one or more of the preselected criteria matched with the criteria used to organize the groups in the original clinical trial data. In some embodiments, the preselected criteria are the criteria from the randomization protocol used in organizing the data structure into the original groups. At 916, multiple treatment allocations are generated based on the reorganization of the groups.

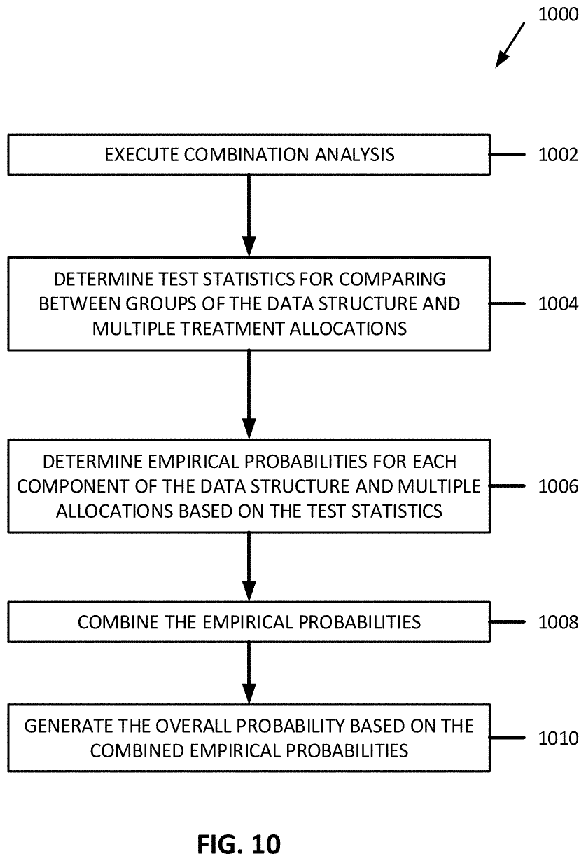

FIG. 10 shows a flow diagram of an illustrative process 1000 for generating overall probabilities, in accordance with some embodiments of the present disclosure. At 1002, combination analysis is executed in order to generate an overall probability based on treatment allocations of the clinical trial data. At 1004, test statistics are determined for comparing each variable between groups of each treatment allocation. For example, medians of each group may be compared. For example, the test statistics may include differences in the medians between groups. At 1006, empirical probabilities are determined for each variable based on the test statistics. For example, for a test statistic corresponding to a component of a first treatment allocation, a p-value may be determined based on the probability of the test statistic being less than or equal to the test statistic in every other treatment allocation. At 1008, the empirical probabilities are combined for each treatment allocation to generate combined empirical probabilities for each treatment allocation. The empirical probabilities may be combined by applying a combining function (e.g., a standard Fisher's function). At 1010, an overall probability, for the clinical trial data, is generated based on the combined empirical probabilities. The overall probability may be the probability of the observed data being less than or equal to the test statistic in every other treatment allocation.

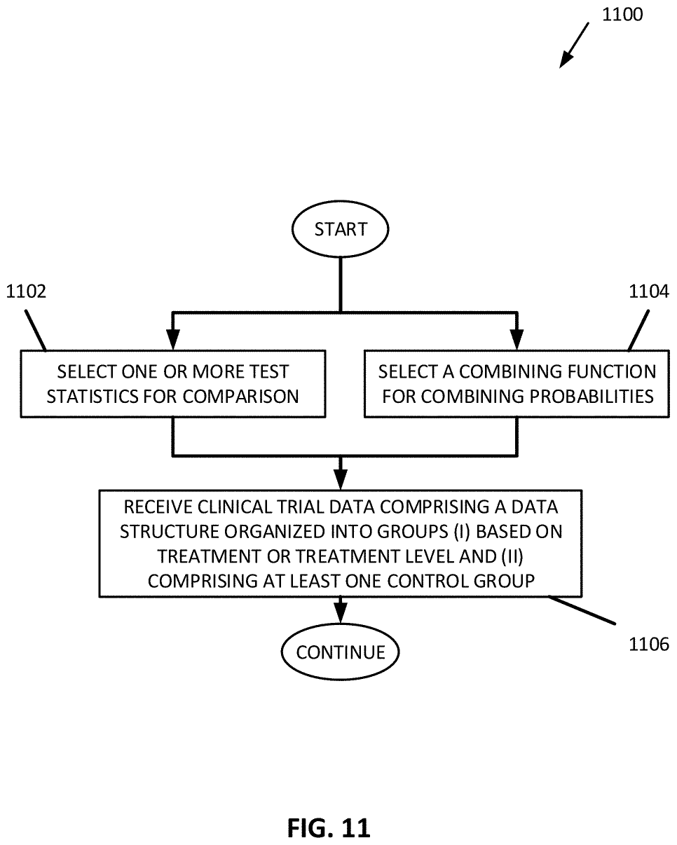

FIG. 11 shows a flow diagram of an illustrative process 1100 for analyzing clinical trial data, in accordance with some embodiments of the present disclosure. At 1102, a test statistic is selected for comparison between groups. At 1104, a combining function is selected for combining probabilities. At 1106, after selecting the test statistic at 1102 and/or selecting the combining function at 1104, clinical trial data are received and continue to another process as described in the present disclosure. For example, process 1100 may be part of process 800, where 1106 is 802. The test statistic and/or combining function may be selected prior to receiving clinical trial data at 802.

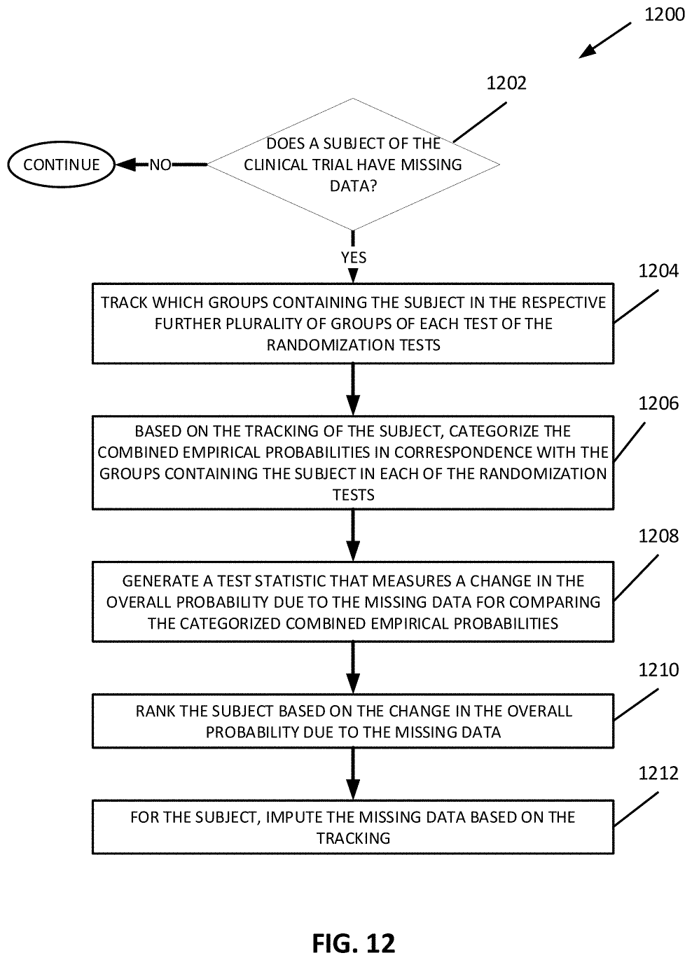

FIG. 12 shows a flow diagram of an illustrative process 1200 for processing missing clinical trial data associated with a subject, in accordance with some embodiments of the present disclosure. At 1202, a subject is checked to determine whether the subject has missing clinical trial data. If the subject does not, process 1200 continues to any other process as described in the present disclosure. If the subject has missing data, process 1200 continues to 1204. At 1204, the groups containing the subject are tracked for each treatment allocation. At 1206, the combined empirical probabilities are categorized based on the tracking of the subject. For example, the combined empirical probabilities are categorized in correspondence with the groups containing the subject for each treatment allocation. At 1208, a test statistic is generated to measure the change in the overall probability due to the missing data of the subject, the test statistics being selected to compare the categorized combined empirical probabilities. At 1210, the subject is ranked among other subjects with missing data. The subject may be ranked based on the change in the overall probability. For example, the change in the overall probability may be due, at least in part, to the missing data associated with the subject. At 1212, the missing data are imputed for the subject (e.g., using multiple imputation) based on the tracking of the subject. For example, the missing data may be inferred based on the groups containing the subject in each treatment allocation.

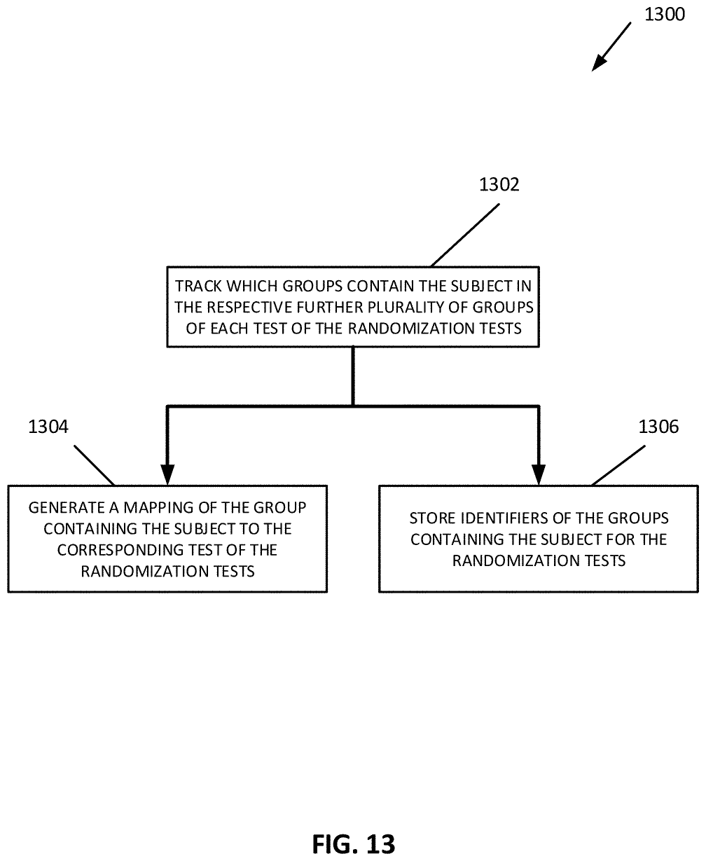

FIG. 13 shows a flow diagram of an illustrative process 1300 for tracking groups containing the subject associated with the missing clinical trial data, in accordance with some embodiments of the present disclosure. At 1302, the groups containing the subject are tracked in each treatment allocation. At 1304, a mapping is generated for the groups containing the subject. For example, a binary matrix may be generated that tracks which treatment allocations placed the subject in which group. At 1306, additionally or alternatively, identifiers of the groups containing the subject for each treatment allocation are stored (e.g., in memory). For example, the treatment allocations may be stored in memory and, for each treatment allocation, the group containing the subject is identified.

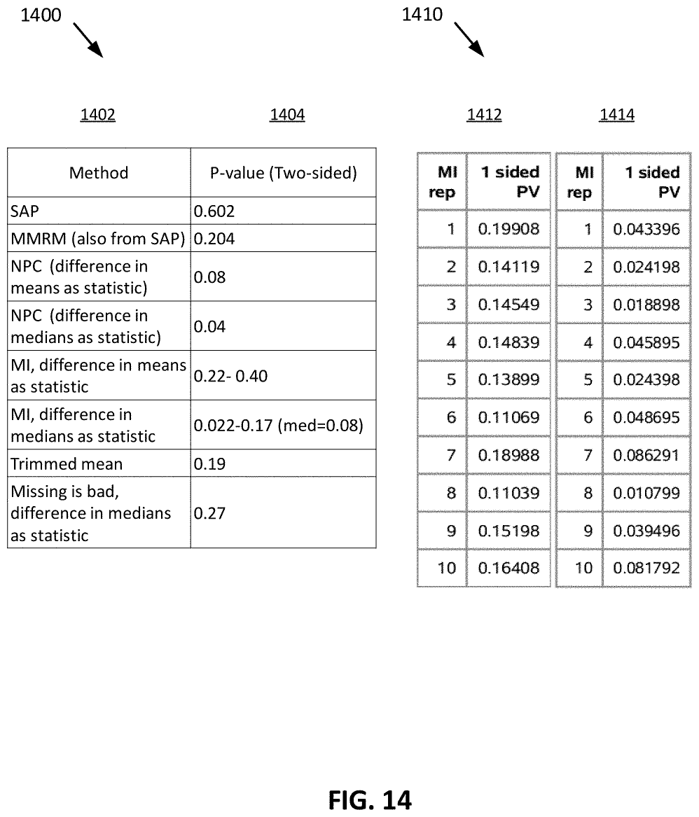

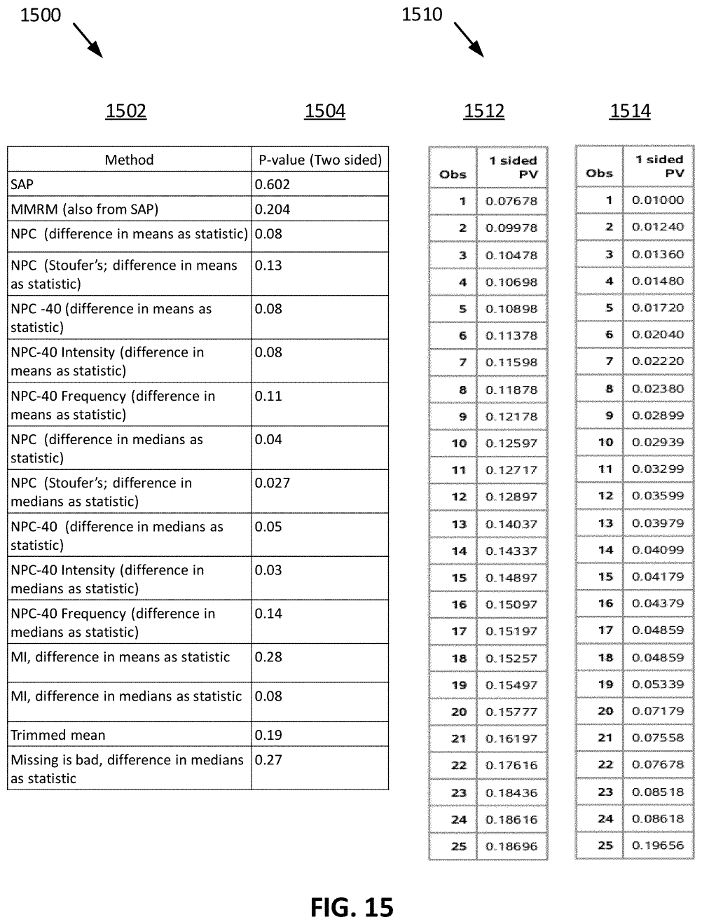

FIGS. 14-15 shows example tables 1400-1410 and 1500-1510 comparing illustrative metrics of randomization tests in accordance with some embodiments of the present disclosure. Results in FIGS. 14 and 15 are based on clinical trial data from a completed twelve-week clinical trial (labeled P301) assessing treatment of military-related PTSD using TNX-102 SL. TNX-102 SL (i.e., TNX) is a cyclobenzaprine HCL-mannitol eutectic tablet designed for sublingual administration and contains a basifying agent. The clinical trial data included subjects' responses to a CAPS-5 questionnaire of twenty questions that independently measure intensity and frequency. Under standard practice, intensity and frequency are typically added into a single measure of severity, which loses information. Instead, intensity and frequency may be considered separately using the randomization tests as shown in FIGS. 14-15. The following includes results from a standard parametric approach for comparison (labeled SAP). FIG. 14 shows illustrative metrics for variants of a randomization test. Table 1410 includes results based on severity (i.e., a dimensionality of 20). At table 1400, column 1402 identifies different variants of a randomization test (labeled NPC at column 1402). At column 1404, illustrative p-values are shown for comparing SAP and variants of the randomization test. Table 1410 includes results using the separated measures of intensity and frequency (i.e., a dimensionality of 40). At table 1410, columns 1412-1414 show illustrative p-value results of randomization tests after the indicated number of multiple imputations (MI). FIG. 15 shows illustrative metrics of randomization tests. At table 1500, column 1502 identifies variants of randomization tests (labeled NPC in 1502). At column 1504, illustrative p-values are shown in comparison with various techniques. At table 1510, columns 1512-1514 show illustrative p-value results of randomization tests after the indicated number of multiple imputation (MI).

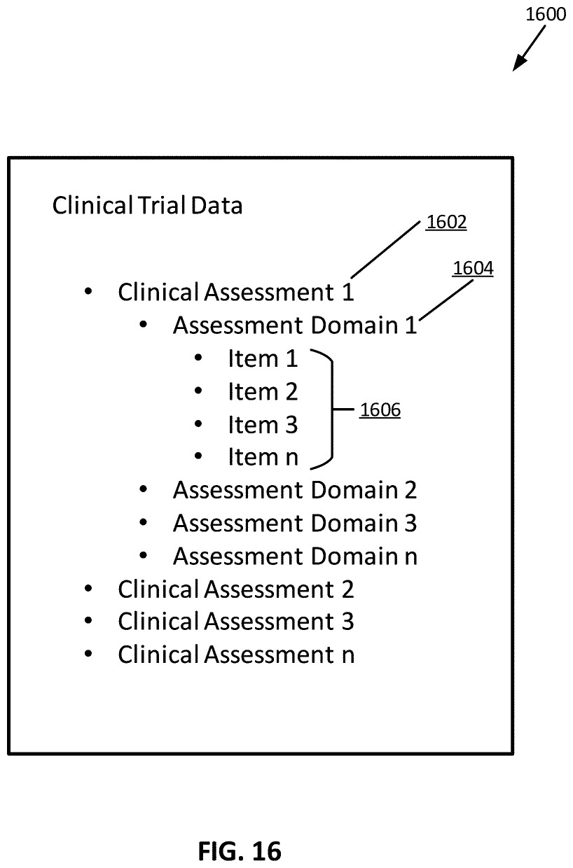

FIGS. 16-24 show illustrative stages that may be part of randomization tests, in accordance with some embodiments of the present disclosure. In some embodiments, clinical trial data may include mixed data from a number of clinical assessments about the health of one or more subjects. Referring now to FIG. 16, data structure 1600 includes illustrative mixed data. Some assessments may include multiple assessment items and may be organized into assessment domains with corresponding metrics (e.g., clinical assessment scales). For example, assessment 1602 includes domain 1604. Domain 1604, in turn, includes items 1606. It should be noted that data structure 1600 illustrates one possible organization and may include different organizations of data. Data structure 1600 may include data from a single assessment or possibly for a single item. The randomization tests may still be used for analyzing such data. Randomization tests may be used to analyze the mixed data from the clinical assessments at different levels of organization. In a non-limiting example, randomization tests may be used for analyzing clinical trial data at each domain, items within a domain, different clinical assessments, or some combination thereof.

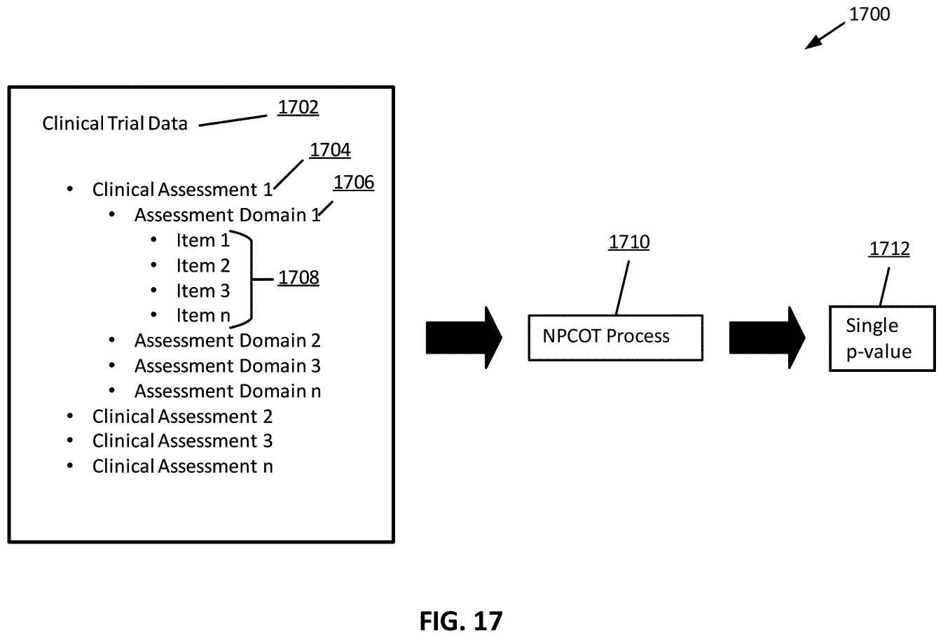

In some embodiments, items from a clinical assessment are analyzed using randomization tests to determine an overall probability (e.g., a statistical p-value) based on comparing subject groups. For example, the subject groups may be treatment and control groups within a clinical trial. Referring now to FIG. 17, a data structure including clinical trial data 1702 is provided. At 1710, NPCOT is used as an illustrative process to analyze clinical trial data 1702. At 1712, a single p-value is generated using NPCOT.

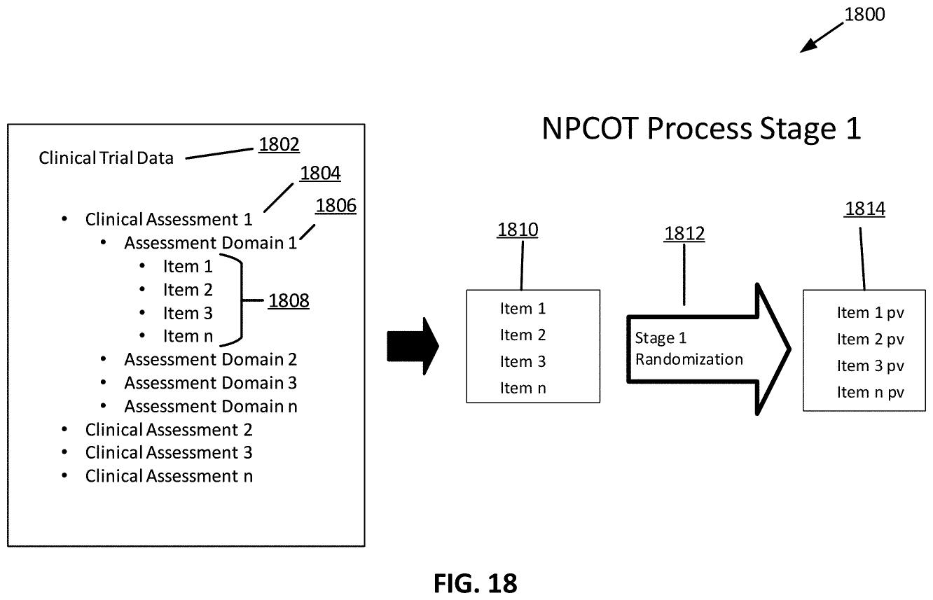

FIG. 18 shows a first stage of an illustrative NPCOT process, in accordance with some embodiments of the present disclosure. In some embodiments, randomization tests include two stages at one or more levels of organization to determine the overall probability. A first stage may include generating allocations as described in the present disclosure as part of a first randomization test. For example, clinical trial data may be allocated at a level of organization (e.g., allocating the data at the level of items) to generate a data structure of empirical probabilities (e.g., a vector of p-values). Items 1810 from clinical trial data 1802 are provided. Items 1810 may be items 1808 or, alternatively, may be items from a different domain and/or different assessment. At 1812, the items are randomized into treatment allocation groups. Empirical probabilities 1814 (e.g., p-values) are generated for each of items 1810.

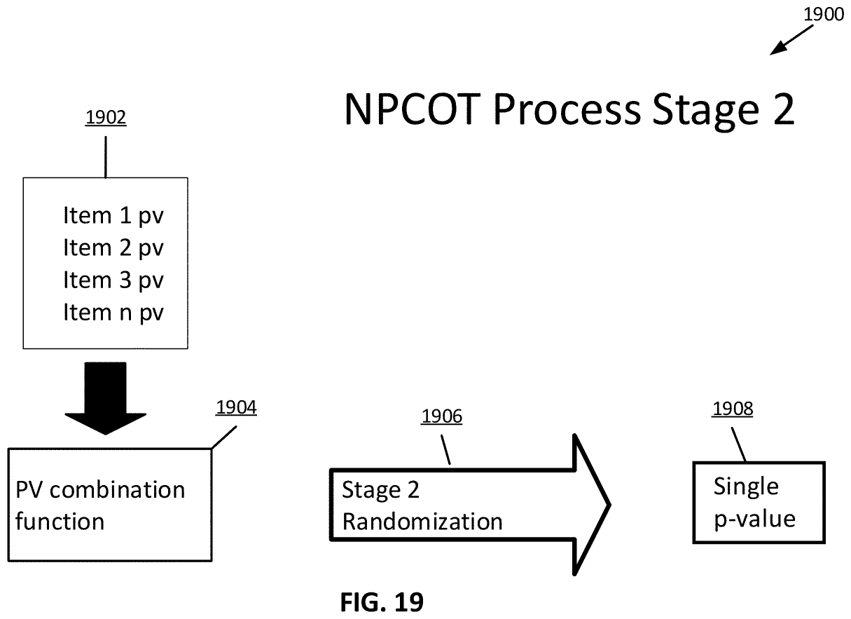

FIG. 19 shows a second stage of an illustrative NPCOT process, in accordance with some embodiments of the present disclosure. In some embodiments, a second stage, as part of a randomization test, may include combining the empirical probabilities (e.g., using a combining function). For example, empirical probabilities 1902 for each assessment item are provided. Empirical probabilities 1902 may be empirical probabilities 1814. Empirical probabilities 1902 are combined using combination function 1904. In some embodiments, the second stage, as part of a randomization test, may include generating the overall probability based on the combined empirical probabilities. In some embodiments, the second stage, as part of a randomization test, may include a second randomization test to generate the overall probability based on the combined empirical probabilities. For example, at 1906, a second randomization is performed using the combined empirical probabilities from 1904 to generate overall probability 1908 (e.g., a single p-value). In some embodiments, a randomization test includes determining one or more test statistics for comparing the groups. An example test statistic, in some preferred embodiments, may include or be a median of each group and/or the difference between the medians. Another example test statistic may include or be a mean. In some embodiments, test statistics may be determined for each assessment item and for each allocation.

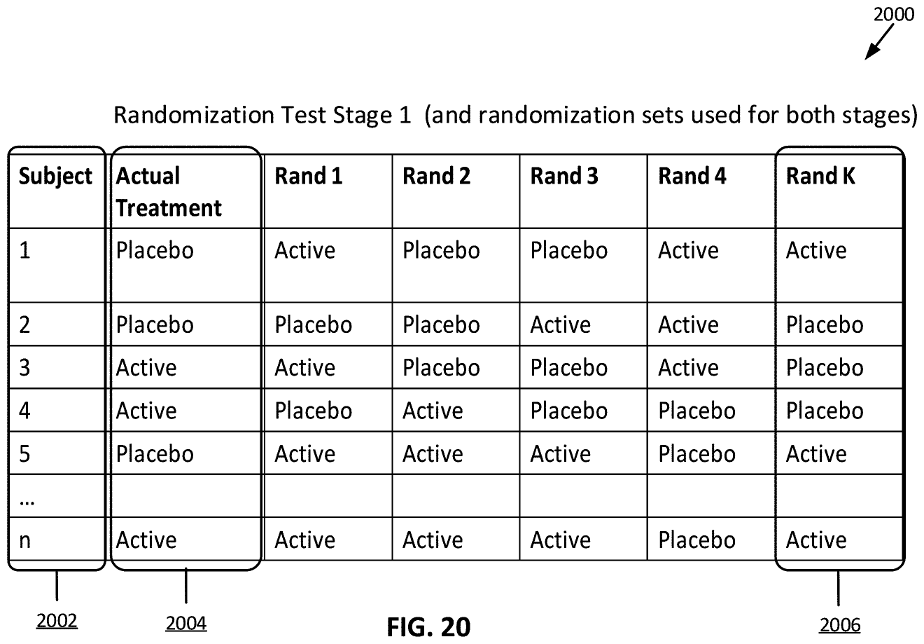

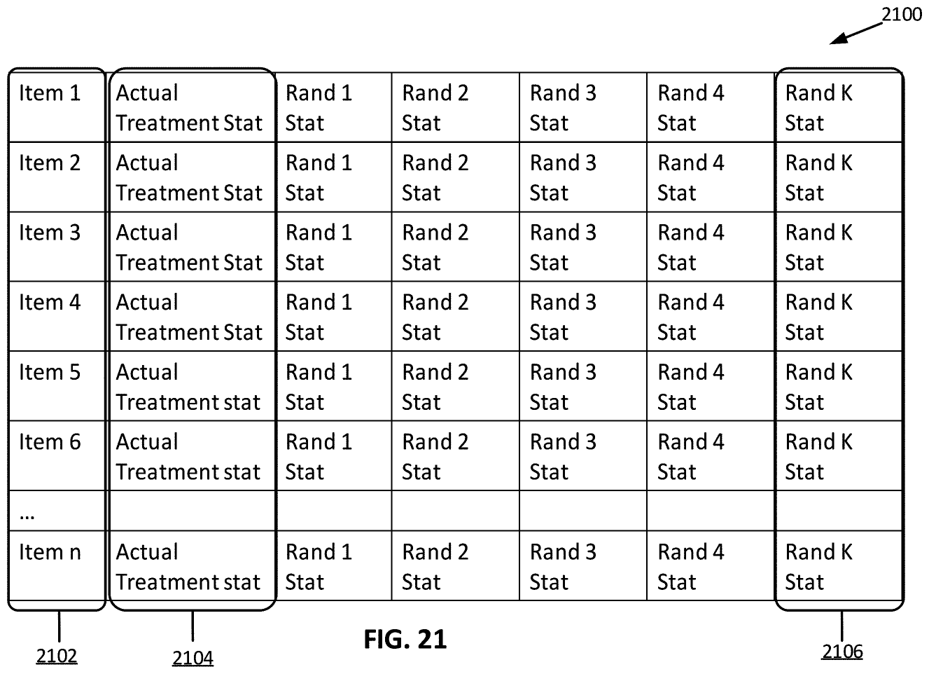

FIG. 20 shows an illustrative data structure 2000 including subjects 2002 with corresponding data, in accordance with some embodiments of the present disclosure. Each of subjects 2002 has originally assigned treatment group 2004. Data structure 2000 includes illustrative random allocations of the subjects into groups (e.g., at an example iteration 2006). FIG. 21 shows an illustrative data structure 2100 containing assessment items 2102, in accordance with some embodiments of the present disclosure. Each of items 2102 has corresponding test statistics 2104 based on the original clinical trial data (e.g., clinical trial data 1702). Data structure 2100 includes test statistics for each of multiple randomized allocations (e.g., example iteration 2106).

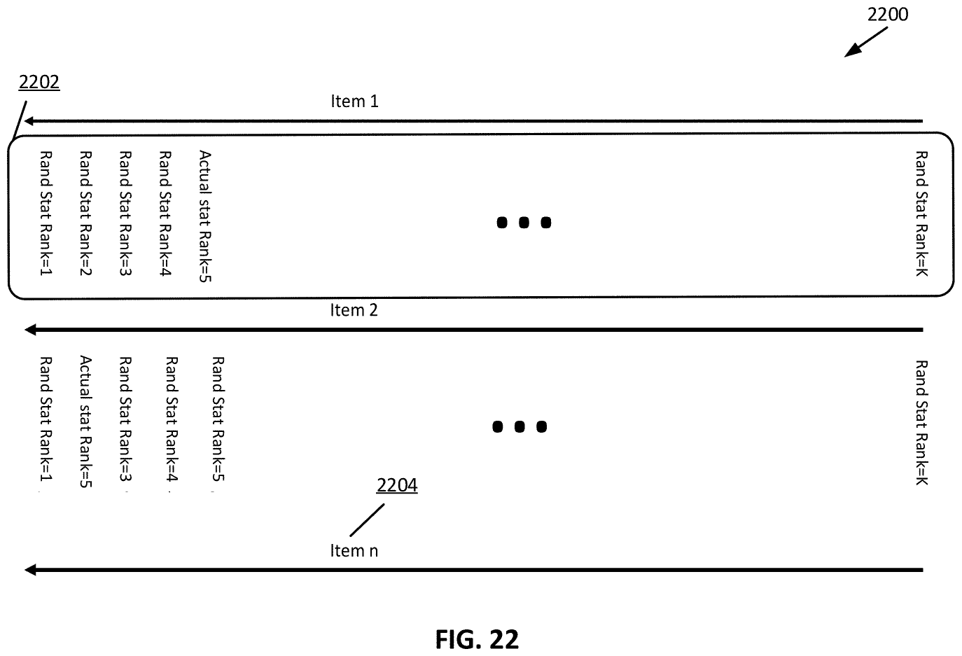

FIG. 22 shows an illustrative data structure 2200 of ranked test statistics to determine empirical probabilities, in accordance with some embodiments of the present disclosure. In some embodiments, the test statistics are ranked. For example, the test statistics may be ranked based on degree of support for a studied treatment (e.g., how well a result supports the studied active component of a drug). At 2202, the test statistics are ranked for a first item up to last item 2204. As a non-limiting example, a statistical p-value may be calculated based on the rank of the results with respect to the observed data divided by the number of allocations (e.g., number of generated allocations plus the observed data). In some embodiments, more allocations are generated to improve precision of an overall probability. It should be noted that the precision of the overall probability is expected to be limited based on the number of allocations. For example, with 99 allocations, an expected smallest p-value may be 0.01.

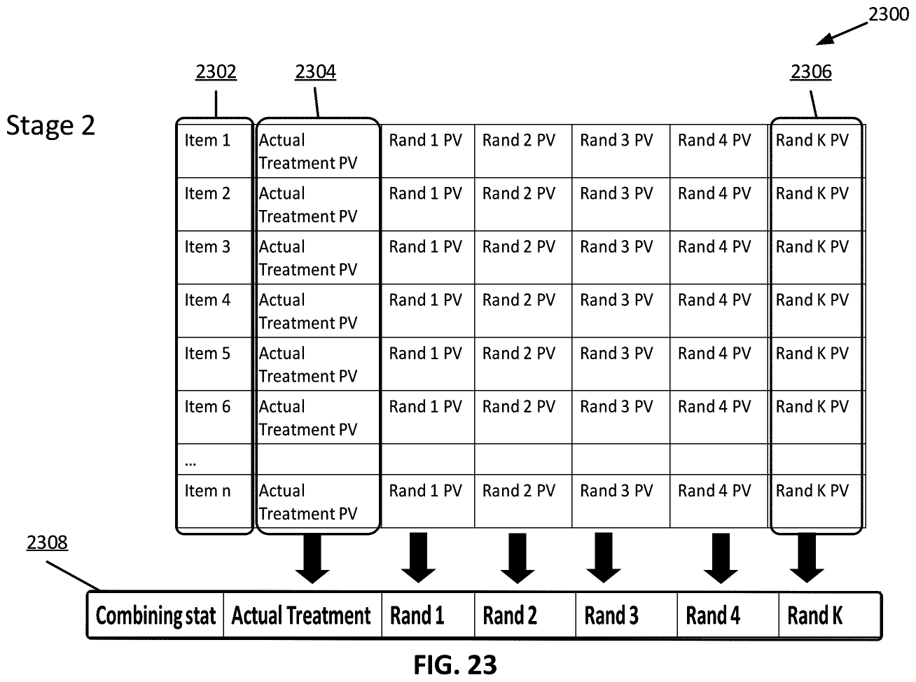

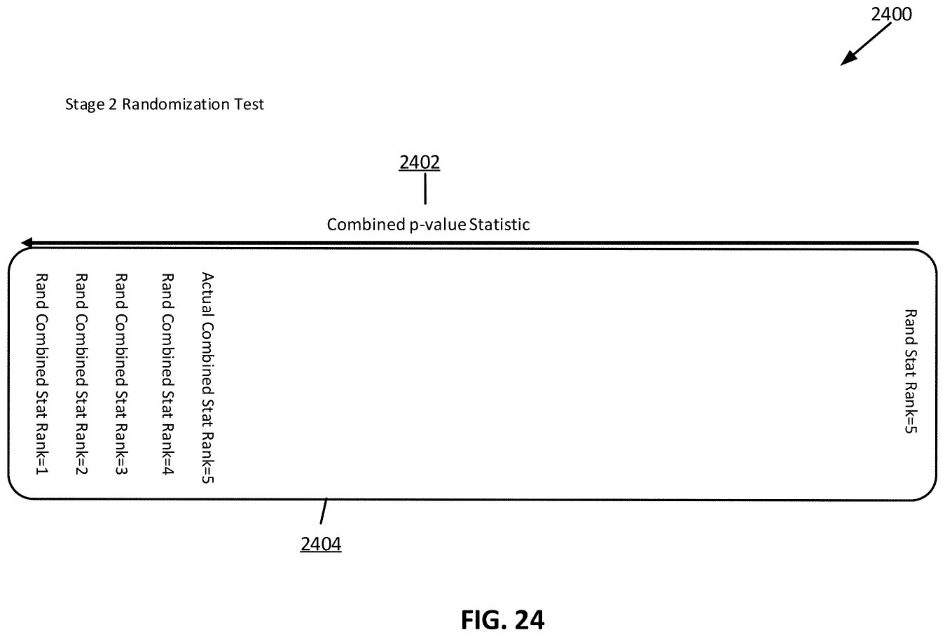

FIG. 23 shows an illustrative data structure 2300 where empirical probabilities are combined as part of a second stage, in accordance with some embodiments of the present disclosure. At 2302-2306, the empirical probabilities are combined for all the items (e.g., using combining function 1904). Combined empirical probabilities 2308 are generated for each randomized allocation. FIG. 24 shows an illustrative data structure 2400 including ranked combined empirical probabilities 2404, in accordance with some embodiments of the present disclosure. In some embodiments, the combined empirical probabilities are ranked. In preferred embodiments, the combined empirical probabilities are ranked similar to how the test statistics are ranked.

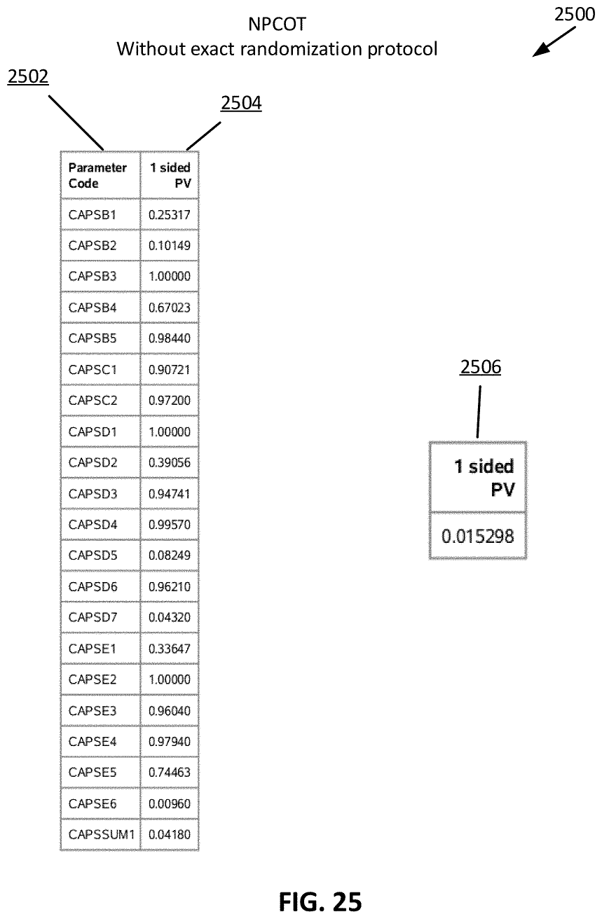

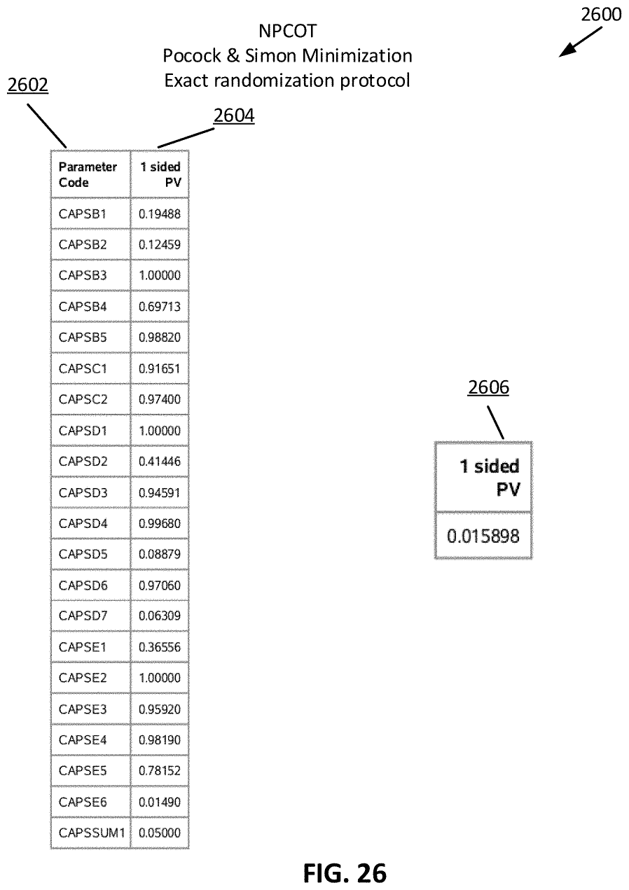

FIGS. 25-26 show example tables comparing illustrative metrics of a randomization test without and with the same randomization protocol from originally assigning subjects to treatment groups, in accordance with some embodiments of the present disclosure. At 2500, NPCOT is used without the same randomization protocol. Column 2502 lists various clinical assessment items along with corresponding p-values at column 2504. At 2506, an overall statistical p-value is shown based on column 2504. At 2600, NPCOT is used with the same randomization protocol and an applied minimization. Column 2602 lists the clinical assessment items along with corresponding p-values at column 2604. At 2606, an overall statistical p-value is shown based on column 2604 to compare against overall statistical p-value at 2506.

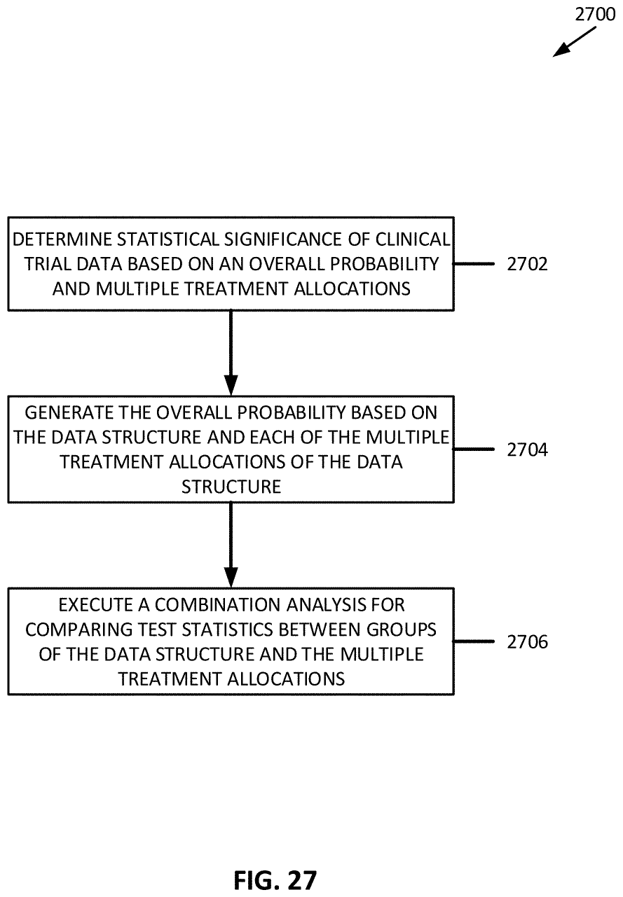

FIG. 27 shows a flow diagram of an illustrative process 2700 for determining statistical significance of results based on clinical trial data, in accordance with some embodiments of the present disclosure. At 2702, statistical significance of results based on the clinical trial data is determined based on an overall probability and the multiple treatment allocations as part of a randomization test. Randomization tests may assess the statistical significance of results based on the clinical trial data based on the multiple treatment allocations without including distributional assumptions that characterize a parametric statistical analysis. In some embodiments, the non-parametric analysis system generates sufficiently many treatment allocations to achieve an expected precision, power, and/or statistical level of significance. At 2704, an overall probability generated based on the data structure and each of the multiple treatment allocations of the data structure (e.g., when determining statistical significance of results based on clinical data). For example, the overall probability may include or be a statistical p-value. At 2706, a combination analysis is executed as part of a randomization test (e.g., when generating the overall probability) to construct an overall test statistic from test statistics for individual dimensions of measurement and to determine an overall probability using the multiple treatment allocations. Constructing the overall test statistic may include comparing test statistics between different groups of the first data structure and/or may include comparing test statistics between different groups of the multiple treatment allocations. In some embodiments, the non-parametric analysis system executes the combination analysis more than once at different times. For example, randomization tests may include executing the combination analysis for data collected at different epochs and/or after different treatment durations to measure the changes in a given response over time. Some important times include but are not limited to: (a) before the trial, (b) at key milestones during the trial (e.g., at beginning, midpoint, and at end of the trial), and (c) at various times after the trial.

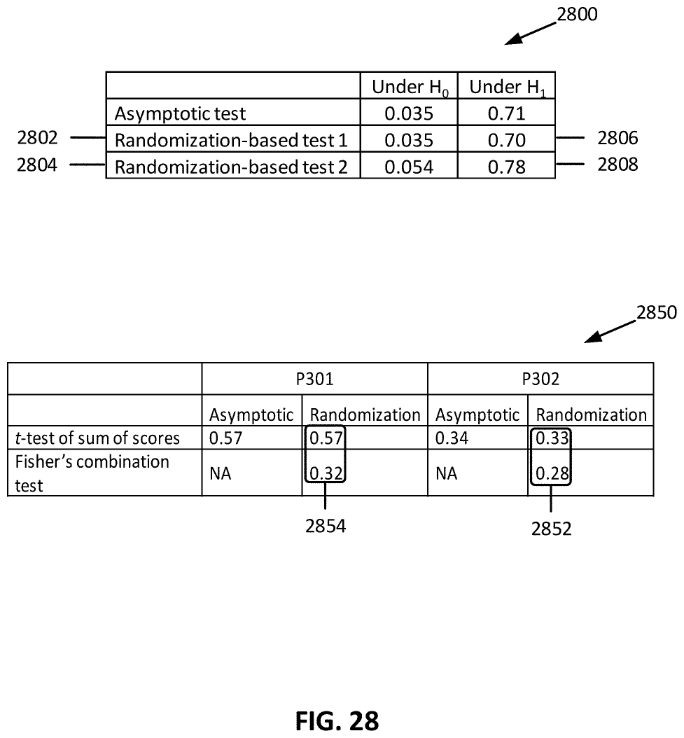

Multiple imputation may be included as part of the NPCOT combined with various embodiments of the present disclosure to address a data set with missing data as described in relation to FIGS. 5-7 and 12 and illustrated in the following examples in reference to FIG. 28. It should be noted that the examples illustrate one combination and there may be other combinations with embodiments of the present disclosure to impute the missing data. In this example, a first data set with a portion of missing data may have been collected from a clinical trial. As part of the NPCOT, multiple imputation may be employed to impute the portion of missing data in the first data set. A simulated distribution for evaluating statistical significance may be generated using the imputed data set. Preferably, the simulated distribution is generated based on the imputed data set and randomized treatment allocations of the imputed data set (e.g., as part of the NPCOT). By using the randomized treatment allocations, the simulated distribution includes additional information that represents the full distribution sampled by the imputed data set.

Results from a simulation study to compare the approaches against an asymptotic t-test involving a sample of 350 participants are provided in Table 2800 (FIG. 28). Table 2800 compares the rejection rate under Ho and Hi for an asymptotic t-test and two randomization-based tests with multiple imputation. At 2802, randomization-based test 1 refers to one embodiment in which the simulated distribution is generated based on randomized treatment allocations of the imputed data set. At 2804, randomization-based test 2 refers to an alternative embodiment in which the simulated distribution is generated based on the imputed data set.

In the aforementioned simulation study, the response data was normally distributed with mean 0.3 in the active treatment group and 0 in the control group, with a common standard deviation of 1. The total sample size was 350 with 175 in each group. The sample size yields a power of 88% when there are no missing data with type I error one-sided 0.05 (denoted Hi). In the simulated study, there was no missing data in the control group (denoted n₁=0). A random number (denoted n₂) of subjects were missing the primary outcome in the active group and generated as a binomial random variable with parameters 175 and 0.2. In this simulation study, missing values were imputed by sampling from the standard normal distribution. Simulation results are based on 25 imputations (denoted m), 1000 allocations (denoted K), and 10000 simulation runs for each set up. As shown at 2806 and 2808, in the embodiment illustrated by the randomization-based test 1 shows behavior similar to the asymptotic t-test whereas the alternative embodiment illustrated by the randomization-based test 2 has an inflated type I error rate. As demonstrated below, either embodiment may be applied to control the type I error at various stages of the NPCOT.

A simulated distribution based on randomized treatment allocations of imputed data may be used at one or more stages of the NPCOT to control the type I error. For example, a combining function (e.g., Fisher, Stouffer, etc.) may be calibrated using a randomization-based test assessed based on statistical significance from the simulated distribution. Calibrating the combining function may control one or more types of statistical errors (e.g., a type I error of the null hypothesis). For example, a data set based on a multi-item assessment may include an item score that is dependent on one or more other item scores, which may inflate the type I error. The calibrated combining function may be less sensitive to errors due to such item dependencies.

As an illustrative example, some results are shown at Table 2850 from a significance assessment including the aforementioned approach. The assessment is based on two randomized clinical trials on PTSD (denoted TNX-CY-P301 and TNX-CY-P302 or P301 and P302, respectively). Table 2850 (FIG. 2850) compares p-values for two PTSD trials, P301 and P302, based on a t-test on a sum of the multi-item scores and a randomization-calibrated Fisher's combination test applied to the same multi-item scores.

P301 was a Phase 3, multicenter, double-blind, placebo-controlled trial conducted in the US in 2017-2018. The members of active treatment group, treated with the investigational drug, TNX-102 SL, were compared to those receiving a placebo. The assessment was conducted on a sample of 252 participants who had experienced PTSD based on the CAPS-5 during military service and had a minimum baseline severity score of >33 on the CAPS-5. 46 participants were missing a primary outcome in P301 out of the 252 participants. The missing data were imputed using multiple imputation in a multi-stage approach. First, intermittent missing values were imputed assuming missing at random within the treatment group and conditioned on the non-missing values, site, sex, tobacco use, and the presence of current a major depressive episode using m=10 imputations and Markov Chain Monte Carlo (MCMC). Next, missing values for subjects who dropped out for atypical reasons were imputed with the same covariates. Finally, missing values for subjects dropping out for typical reasons were imputed from the baseline distribution pooled across the treatment groups and conditioned on the non-missing post-baseline values. The same randomization design was used to obtain K=5000 additional random assignment vectors for the randomization-calibrated Fisher's combination test.

P302 was a multicenter, double-blind, placebo-controlled trial Phase 3 study of TNX-102 SL in PTSD. In P302, the inclusion criteria were broadened, as compared to P301, to include civilian participants with active PTSD (as determined by the CAPS-5) who experienced an index trauma within nine years of screening. P302 included 94% civilian trauma with a minimum baseline severity score of ≥33 on the CAPS-5. There were 36 subjects missing a primary outcome in P302. The imputation approach was identical to that of P301 except that the variables were limited to site, sex, and the non-missing CAPS-5 values. Permuted block design with blocks of size 2 and 4 within each of the 28 recruiting sites was used to randomize participants in P302. The same randomization design was used to obtain K=5000 permuted assignment vectors for randomization-based tests.

As shown in Table 2850 (FIG. 28), the nonparametric test using Fisher's combining function yields a smaller p-value than the t-test because the treatment primarily affected only a subset of the scores. A randomization-based combination test may be combined with multiple imputation (e.g., as described in relation to NPCOT) to account for missing data in a robust manner. At 2852, the randomization-based combination test has similar behavior as a t-test when treatment improves all items. At 2854, the randomization-based combination test shows improved behavior when the treatment substantially improves a small subset of items.

Any of the systems and methods in their various embodiments as described in the present disclosure may be used including, but not limited, for various products, pursuits, and/or endeavors. In some embodiments, a pharmaceutical or biologic agent or composition may be characterized in that the agent's approval for marketing is based on an application that is characterized at least in part by an assessment of the statistical significance and/or an efficacy and safety of the agent, composition, treatment, or combination based on clinical trial data from a clinical trial of the agent using any of the systems and methods described in the present disclosure. In some embodiments, an application for marketing approval of a pharmaceutical or biologic agent and/or a use thereof may be composed and/or submitted based on any of the systems and methods described in the present disclosure. For example, the application may be characterized at least in part by an assessment of the statistical significance and/or an efficacy and safety of the agent, composition, treatment, or combination based on clinical data from a clinical trial of the agent using any of the systems and methods described in the present disclosure. In some embodiments, a pharmaceutical or biologic agent or composition thereof may be marketed based on any of the systems and methods described in the present disclosure. For example, marketing a pharmaceutical or biologic agent or composition thereof may be characterized in that the agent's approval for marketing was based on an application that is characterized at least in part by an assessment of the statistical significance and/or an efficacy and safety of the agent, composition, treatment, or combination based on clinical trial data from a clinical trial of the agent using any of the systems and methods described in the present disclosure.

In some embodiments, a pharmaceutical or biologic agent, a composition of one or both, a non-pharmaceutical or non-biologic treatment of a disorder, disease, or condition, or a combination of any thereof may be characterized in that the approval of the agent, composition, treatment, or combination for marketing at least in part, relates to or depends on an assessment of the statistical significance and/or an efficacy and safety of the agent, composition, treatment, or combination based on clinical trial data from a clinical trial of the agent, composition, treatment, or combination determined using any of the systems and methods described in the present disclosure.

In some embodiments, an application for marketing approval, (e.g., of a pharmaceutical or biologic agent, a composition of one or both, and/or a use thereof, a non-pharmaceutical or non-biologic treatment of a disorder, disease, or condition, or a combination of any of them) may be characterized at least in part by an assessment of the statistical significance and/or an efficacy and safety of the agent, composition, treatment, or combination based on clinical data from a clinical trial of the agent, composition, treatment, or combination determined, at least in part, using any of the systems and methods described in the present disclosure.

In some embodiments, selling, offering for sale, or importing a pharmaceutical or biologic agent, a composition of one or both, a non-pharmaceutical or non-biologic treatment of a disorder, disease or condition, or a combination of any of them may be characterized in that approval of the agent, composition, treatment or combination for marketing, at least in part, relates to or depends on an assessment of the statistical significance and/or an efficacy and safety of the agent, composition, treatment, or combination based on clinical trial data from a clinical trial of the agent, composition, treatment or combination determined, at least in part, using any of the systems and methods described in the present disclosure.

In some embodiments, marketing a pharmaceutical or biologic agent or composition thereof may be characterized in that the agent's approval for marketing was based on an application that is characterized at least in part by an assessment of the statistical significance and/or an efficacy and safety of the agent, composition, treatment, or combination based on clinical trial data from a clinical trial of the agent using any of the systems and methods described in the present disclosure.

In some embodiments, a pharmaceutical or biologic agent, a composition of one or both, a non-pharmaceutical or non-biologic treatment of a disorder, disease, or condition or a combination of any of them may be characterized in that approval of the agent, composition, treatment or combination for marketing, at least in part, relates to or depends on an assessment of the statistical significance and/or an efficacy and safety of the agent, composition, treatment, or combination based on data that assess one or both of the bioequivalence or lack of inferiority of the agent, composition, treatment, or combination as compared to an existing agent, composition, treatment or combination, wherein assessing the statistical significance and/or the efficacy and safety of the agent, composition, treatment, or combination based on the data uses, at least in part, any of the systems and methods described in the present disclosure.

In some embodiments, one or both of the bioequivalence or lack of inferiority is assessed for a pharmaceutical or biologic agent, a composition of one or both, and/or a use thereof, a non-pharmaceutical or non-biologic treatment of a disorder, disease, or condition, or a combination of any of them as compared to an existing agent, composition, use, treatment or combination of any of them, including assessing the statistical significance and/or an efficacy and safety of the agent, composition, treatment, or combination based on data related to one or both of the bioequivalence or lack of inferiority wherein assessing the statistical significance and/or the efficacy and safety of the agent, composition, treatment, or combination based on the data uses, at least in part, any of the systems and methods described in the present disclosure.

In some embodiments, an application may be submitted for marketing approval of a pharmaceutical or biologic agent, a composition of one or both, and/or a use thereof, a non-pharmaceutical or non-biologic treatment of a disorder, disease, or condition, or a combination of any of them, the application being characterized at least in part by an assessment of the statistical significance and/or an efficacy and safety of the agent, composition, treatment, or combination based on data related to one or both of the bioequivalence or lack of inferiority of the agent, composition, treatment, or combination as compared to an existing agent, composition, treatment or combination of any of them, wherein assessing the statistical significance and/or the efficacy and safety of the agent, composition, treatment, or combination based on the data uses, at least in part, any of the systems and methods described in the present disclosure.

In some embodiments, selling, offering for sale, or importing a pharmaceutical or biologic agent, a composition of one or both, a non-pharmaceutical or non-biologic treatment of a disorder, disease or condition, or a combination of any of them may be characterized in that approval of the agent, composition, treatment of combination for marketing at least in part relates to or depends on an assessment of the statistical significance and/or an efficacy and safety of an agent, composition, treatment, or combination based on data related to one or both of the bioequivalence or lack of inferiority of the agent, composition, treatment or combination as compared to an existing agent, composition, treatment or combination of any of them, wherein assessing the statistical significance and/or an efficacy and safety of the agent, composition, treatment, or combination based on the data uses, at least in part, any of the systems and methods described in the present disclosure.

In some embodiments, efficacy and safety of an agent, composition, treatment, or combination based on statistical significance is assessed for one of more toxicities or adverse events or a combination thereof observed after administration to a subject of a pharmaceutical or biologic agent, or a composition of one or both, or after treating a subject for a disease, disorder or condition, with a non-pharmaceutical or non-biologic therapy or a combination of them, including assessing the statistical significance and/or the efficacy and safety of the agent, composition, treatment, or combination based on the one of more toxicities or adverse events using, at least in part, any of the systems and methods described in the present disclosure.

The foregoing is merely illustrative of the principles of this disclosure and various of its embodiments. Various modifications may be made by those skilled in the art without departing from the scope of this disclosure. The above-described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations and modifications thereof, which are within the spirit of the following exemplary embodiments and claims.

### Exemplary Embodiments

1. A method for an efficacy and safety of an agent, composition, treatment, or combination based on clinical trial data, the method comprising:

receiving clinical trial data, wherein the clinical trial data comprise a data structure comprising data corresponding to subjects in the clinical trial, wherein the subjects have been organized into a plurality of groups based on treatments or treatment levels, and wherein at least one group of the plurality of groups is a control group; and

generating multiple treatment allocations of the data structure, wherein generating the multiple treatment allocations comprises, for each of the multiple treatment allocations, reorganizing, at random, the subjects along with the corresponding data to generate further pluralities of groups without regard to the respective treatment, treatment level, or status as the control group in the data structure.

2. The method of embodiment 1, wherein the multiple treatment allocations are generated at random without constraints or balancing criteria.
3. The method of embodiment 1, wherein, for each multiple treatment allocation, a probability for a subject being reorganized into a group of the further pluralities of groups is comparable to a probability of the subject having been organized into a group of the plurality of groups based on the treatments or the treatment levels.
4. The method of embodiment 1, wherein the subjects are reorganized to generate the further pluralities of groups while maintaining group sizes appropriate for assessing the efficacy and safety of treatments based on the received clinical trial data.
5. The method of embodiment 1, wherein each of the multiple treatment allocations is generated using a randomization protocol.
6. The method of embodiment 1, wherein the randomization protocol honors the randomization design of the clinical trial.
7. The method of embodiment 1, wherein generating the multiple treatment allocations using the randomization protocol comprises executing a group algorithm to form groups of subjects based on preselected criteria for balancing the groups and at least one of a temporal sequence.
8. The method of embodiment 7, wherein the preselected criteria match at least some of criteria from a randomization protocol used for organizing the data structure into the plurality of groups except for the treatment level or a treatment intensity and at least one of a temporal sequence.
9. The method of embodiment 7, wherein the preselected criteria are criteria from a randomization protocol used for organizing the data structure into the plurality of groups except for the treatment level or a treatment intensity and at least one of a temporal sequence.
10. The method of embodiment 7, wherein the preselected criteria comprise, for each of the groups, one or more of a group size, a demographic distribution, gender, age, or ethnicity.
11. The method of embodiment 10, wherein the group size matches the size of each of the plurality of groups based on the treatment levels.
12. The method of embodiment 1, wherein the clinical trial data corresponding to each subject in each group of the plurality of groups depends only on the treatment level of the each group for which the each subject is assigned.
13. The method of embodiment 1, wherein assessing the efficacy and safety of the agent, composition, treatment, or combination based on the clinical trial data does not include distributional assumptions that characterize a parametric approach.
14. The method of embodiment 1, wherein the clinical trial data comprise ordinal data corresponding to each of the subjects, and wherein generating the multiple treatment allocations enables addressing the ordinal data when assessing the efficacy and safety of the agent, composition, treatment, or combination based on the clinical trial data.
15. The method of embodiment 1, further comprising executing one or more sensitivity analyses to determine if a result based on the clinical trial data is due at least in part to a side effect of a treatment.
16. The method of embodiment 15, wherein the side effect comprises a habit-forming effect.
17. The method of embodiment 16, wherein the habit-forming effect comprises an addictive or other reinforcing property.
18. The method of embodiment 1, further comprising executing one or more sensitivity analyses to determine if a result based on the clinical trial data is due at least in part to unblinding the clinical trial.
19. The method of embodiment 1, wherein generating the multiple treatment allocations enables analyzing the clinical trial data without reducing components of the data structure or combining data corresponding to the subjects.
20. The method of embodiment 19, wherein analyzing the clinical trial data without reducing the components comprises determining importance weights for the components of the data structure.
21. The method of embodiment 1, wherein the clinical trial data comprise multivariate data corresponding to each of the subjects, and wherein generating the multiple treatment allocations enables analyzing the multivariate data while minimizing information loss.
22. The method of embodiment 21, wherein analyzing the multivariate data while minimizing information loss comprises determining importance weights for variables of the multivariate data.
23. The method of embodiment 21, wherein variables of the multivariate data correspond to multiple criteria, wherein the multivariate data comprise baseline comparison data for the multiple criteria corresponding to subjects in the clinical trial, and wherein the baseline comparison data are based on comparison between data at a first time point before or in the clinical trial and data at a second time point in or after the clinical trial.
24. The method of embodiment 23, wherein the multiple criteria comprise questions about a disorder or condition for the subject, inquiring about one or more of a state, a presence of any symptoms, or a severity of symptoms before, during, and at various timepoints after the clinical trial.
25. The method of embodiment 23, wherein the baseline comparison data comprise values on a discrete scale.
26. The method of embodiment 23, wherein the baseline comparison data comprise categorical responses to the multiple criteria.
27. The method of embodiment 21, wherein the multivariate data comprise correlations among some or all variables.
28. The method of embodiment 1, further comprising:

determining the efficacy and safety of the agent, composition, treatment, or combination based on the clinical trial data based on an overall probability and the multiple treatment allocations of the data structure, wherein:
determining the efficacy and safety of the agent, composition, treatment, or combination based on comprises generating the overall probability based on the data structure and each of the multiple treatment allocations of the data structure; and
generating the overall probability comprises executing a combination analysis for comparing test statistics between each group of the plurality of groups of the data structure and for comparing test statistics between each group of each of the further pluralities of groups of the multiple treatment allocations.
   29. The method of embodiment 28, wherein generating the overall probability, based on the data structure and the multiple treatment allocations, preserves directionality of the overall probability.
   30. The method of embodiment 29, wherein preserving the directionality of the overall probability enables determining a statistical p-value.
   31. The method of embodiment 28, wherein the combination analysis is executed more than once at different times during the clinical trial or after the clinical trial.
   32. The method of embodiment 28, wherein the combination analysis is executed using processing circuitry.
   33. The method of embodiment 28, wherein the overall probability is used to determine whether a null hypothesis is true or false.
   34. The method of embodiment 28, wherein executing the combination analysis comprises:

determining test statistics for comparing between groups in the plurality of groups and between groups in each of the further pluralities of groups, wherein the test statistics correspond to each of the components of the data structure and the multiple treatment allocations;

determining empirical probabilities for each component of the data structure and the multiple treatment allocations based on the test statistics;

combining the empirical probabilities, wherein combining the empirical probabilities comprises applying a combining function to the empirical probabilities; and

generating the overall probability based on the combined empirical probabilities.

35. The method of embodiment 34, wherein the empirical probabilities are determined based on ranking the test statistics.
36. The method of embodiment 34, wherein the test statistics are medians corresponding to the components of the data structure and the multiple treatment allocations.
37. The method of embodiment 34, further comprising, selecting the combining function prior to receiving the clinical trial data.
38. The method of embodiment 37, wherein the selected combining function is a Fisher combining function, a Liptak combining function, or a Stouffer combining function.
39. The method of embodiment 34, wherein a subject of the clinical trial is missing a portion of the clinical trial data associated with one or more of a subject in the clinical trial, further comprising:

tracking which groups contain the subject in the respective further plurality of groups of each treatment allocation of the multiple treatment allocations;

based on the tracking of the subject, categorizing the combined empirical probabilities in correspondence with the groups containing the subject in each of the multiple treatment allocations; and

generating a test statistic for comparing the categorized combined empirical probabilities, wherein the test statistic measures a change in the overall probability due to the missing portion of the clinical trial data associated with the subject.

40. The method of embodiment 39, further comprising ranking the subject based on the change in the overall probability due to the missing portion of the clinical trial data associated with the subject.
41. The method of embodiment 39, further comprising, for the subject, imputing the missing portion of the clinical trial data associated with the subject.
42. The method of embodiment 41, wherein the missing portion is imputed more than once at different times.
43. The method of embodiment 39, wherein tracking which groups contain the subject comprises generating a mapping of the groups containing the subject in the respective treatment allocation of the multiple treatment allocations.
44. The method of embodiment 39, wherein tracking which groups contain the subject comprises storing, in a data structure, identifiers of the groups containing the subject for the multiple treatment allocations.
45. The method of embodiment 39, wherein generating the test statistic comprises applying a transformation to each of the categorized combined empirical probabilities.
46. The method of embodiment 39, wherein generating the test statistic comprises determining a Hodges-Lehmann-Sen estimator.
47. The method of embodiment 1, wherein sufficient treatment allocations are generated to achieve a desired precision or statistical level of significance.
48. The method of embodiment 1, further comprising, selecting one or more test statistics for comparison prior to receiving the clinical trial data.
49. The method of embodiment 1, wherein the treatment level comprises at least one of a treatment intensity, a treatment dosage, and a treatment frequency.
50. The method of embodiment 1, wherein the clinical trial data is data from a clinical trial of a potential agent or other intervention to treat a psychological condition, a psychological syndrome, a psychiatric disorder, or a central nervous system disease.
51. The method of embodiment 50, wherein the potential agent or the other intervention is for treating post-traumatic stress disorder (PTSD).
52. The method of embodiment 1, wherein the method is a computer-implemented method, and wherein the multiple treatment allocations are generated using processing circuitry.
53. The method of embodiment 1, further comprising evaluating results of the clinical trial to estimate parameters for a future clinical study.
54. The method of embodiment 1, further comprising evaluating interim results of the clinical trial to re-estimate at least one of a group size parameter, a treatment intensity, or a treatment dosage in an adaptive clinical trial.
55. The method of embodiment 1, further comprising evaluating safety of a treatment being assessed in the clinical trial.
56. The method of embodiment 55, wherein evaluating the safety of the treatment comprises assessing if a toxicity due to the treatment is observed.
57. The method of embodiment 1, wherein the clinical trial data comprise data based on one or more clinical assessment scales.
58. The method of embodiment 57, wherein the clinical trial data comprise data based on assessment domains of the one or more clinical assessment scales, and wherein each assessment domain comprises a plurality of assessment items.
59. The method of embodiment 1, wherein the clinical trial data comprise data collected from trials at one or more locations, and wherein the plurality of groups are further organized based on the one or more locations.
60. A system for assessing an efficacy and safety of an agent, composition, treatment, or combination based on clinical trial data from a clinical trial, the system comprising:

one or more input/output (I/O) paths for receiving and transmitting data; and

processing circuitry coupled to the one or more I/O paths and configured to:
receive, via the one or more I/O paths, clinical trial data, wherein the clinical trial data comprise a data structure comprising data corresponding to subjects in the clinical trial, wherein the subjects have been organized into a plurality of groups based on treatments or treatment levels, and wherein at least one group of the plurality of groups is a control group; and
generate multiple allocations of the data structure;

wherein the processing circuitry, when generating the multiple allocations, is configured to reorganize, at random for each of the multiple allocations, the subjects along with the corresponding data to generate further pluralities of groups without regard to the respective treatment, treatment level, or status as the control group.

61. The system of embodiment 60, wherein the processing circuitry is configured to generate the multiple treatment allocations at random without constraints or balancing criteria.
62. The system of embodiment 60, wherein, for each multiple treatment allocation, a probability for a subject being reorganized into a group of the further pluralities of groups is comparable to a probability of the subject having been organized into a group of the plurality of groups based on the treatments or the treatment levels.
63. The system of embodiment 60, wherein the processing circuitry is configured to reorganize the subjects to generate the further pluralities of groups while maintaining group sizes appropriate for assessing the efficacy and safety of the agent, composition, treatment, or combination based on the received clinical trial data.
64. The system of embodiment 60, wherein the processing circuitry is configured to generate each of the multiple allocations using a randomization protocol.
65. The system of embodiment 64, wherein the randomization protocol honors the randomization design of the clinical trial.
66. The system of embodiment 64, wherein the processing circuitry, when generating the multiple treatment allocations using the randomization protocol, is configured to execute a group algorithm to form groups of subjects based on preselected criteria for balancing the groups (and at least one of a temporal sequence).
67. The system of embodiment 66, wherein the preselected criteria match at least some of criteria from a randomization protocol used for organizing the data structure into the plurality of groups (and at least one of a temporal sequence) except for the treatment levels or a treatment intensity.
68. The system of embodiment 66, wherein the preselected criteria are criteria from a randomization protocol used for organizing the data structure into the plurality of groups except for the treatment level or a treatment intensity and at least one of a temporal sequence.
69. The system of embodiment 66, wherein the preselected criteria comprise, for each of the groups, one or more of a group size, a demographic distribution, gender, age, or ethnicity.
70. The system of embodiment 69, wherein the group size matches the size of each of the plurality of groups based on the treatment levels.
71. The system of embodiment 60, wherein the clinical trial data corresponding to each subject in each group of the plurality of groups depends only on the treatment level of the each group for which the each subject is assigned.
72. The system of embodiment 60, wherein assessing the efficacy and safety of treatments based on the clinical trial data based on the multiple allocations does not include distributional assumptions that characterize a parametric approach.
73. The system of embodiment 60, wherein the clinical trial data comprise ordinal data corresponding to each of the subjects, and wherein the processing circuitry, when generating the multiple allocations, is configured to address the ordinal data when assessing the efficacy and safety of treatments based on the clinical trial data.
74. The system of embodiment 60, wherein the processing circuitry is further configured to execute one or more sensitivity analyses to determine if a result based on the clinical trial data is due at least in part to a side effect of a treatment.
75. The system of embodiment 74, wherein the side effect comprises a habit-forming effect.
76. The system of embodiment 75, wherein the habit-forming effect comprises an addiction or other reinforcing property.
77. The system of embodiment 60, wherein the processing circuitry is further configured to execute one or more sensitivity analyses to determine if a result based on the clinical trial data is due at least in part to unblinding the clinical trial.
78. The system of embodiment 60, wherein the processing circuitry, when generating the multiple allocations, is configured to analyze the clinical trial data without reducing components of the data structure or combining data corresponding to the subjects.
79. The method of embodiment 78, wherein the processing circuitry, when analyzing the clinical trial data without reducing the components, is configured to determine importance weights for the components of the data structure.
80. The system of embodiment 60, wherein the clinical trial data comprise multivariate data corresponding to each of the subjects, and wherein the processing circuitry, when generating the multiple allocations, is configured to analyze the multivariate data while minimizing information loss.
81. The method of embodiment 80, wherein the processing circuitry, when analyzing the multivariate data while minimizing information loss, is configured to determine importance weights for variables of the multivariate data.
82. The system of embodiment 80, wherein variables of the multivariate data correspond to multiple criteria, wherein the multivariate data comprise baseline comparison data for the multiple criteria corresponding to subjects in the clinical trial, and wherein the baseline comparison data are based on comparison between data at a first time point in the clinical trial and data at a second time point in the clinical trial.
83. The system of embodiment 82, wherein the multiple criteria comprise questions about a disorder or condition for the subject, inquiring about one or more of a state, a presence of any symptoms, or a severity of symptoms before, during, and at various timepoints after the trial.
84. The system of embodiment 82, wherein the baseline comparison data comprise values on a discrete scale.
85. The system of embodiment 82, wherein the baseline comparison data comprise categorical responses to the multiple criteria.
86. The system of embodiment 80, wherein the multivariate data comprise correlations among some or all variables.
87. The system of embodiment 60, wherein the processing circuitry is further configured to determine the statistical significance of results from the clinical trial data based on an overall probability and the multiple allocations of the data structure, and

wherein the processing circuitry is configured to:
when determining the statistical significance, generate the overall probability based on the data structure and each of the multiple allocations of the data structure; and
when generating the overall probability, execute a combination analysis for comparing test statistics between each group of the plurality of groups of the data structure and for comparing test statistics between each group of each of the further pluralities of groups of the multiple allocations.
   88. The system of embodiment 87, wherein the processing circuitry, when generating the overall probability based on the data structure and the multiple allocations, is configured to preserve directionality of the overall probability.
   89. The system of embodiment 88, wherein the processing circuitry, when preserving the directionality of the overall probability, is configured to determine a statistical p-value.
   90. The system of embodiment 87, wherein the processing circuitry is configured to execute the combination analysis more than once at different times of the clinical trial.
   91. The system of embodiment 87, wherein the overall probability is used to determine whether a null hypothesis is false.
   92. The system of embodiment 87, wherein:
the processing circuitry, when executing the combination analysis, is configured to:
   determine test statistics for comparing between groups in the plurality of groups and between groups in each of the further pluralities of groups, wherein the test statistics correspond to each of the components of the data structure and the multiple allocations;
   determine empirical probabilities for each component of the data structure and multiple allocations based on the test statistics;
   combine the empirical probabilities; and
   generate the overall probability based on the combined empirical probabilities; and
the processing circuitry, when combining the empirical probabilities, is configured to apply a combining function to the empirical probabilities.
   93. The system of embodiment 92, wherein the processing circuitry is configured to determine the empirical probabilities based on ranking the test statistics.
   94. The system of embodiment 92, wherein the test statistics are medians corresponding with the components of the data structure and the multiple allocations.
   95. The system of embodiment 92, wherein the processing circuitry is further configured to select the combining function prior to receiving the clinical trial data.
   96. The system of embodiment 95, wherein the selected combining function is a Fisher combining function, a Liptak combining function, or a Stouffer combining function.
   97. The system of embodiment 92, wherein a subject of the clinical trial is missing a portion of the clinical trial data associated with the subject, and wherein the processing circuitry is further configured to:

track which groups contain the subject in the respective further plurality of groups of each allocation of the multiple allocations;

based on the tracking of the subject, categorize the combined empirical probabilities in correspondence with the groups containing the subject in each of the multiple allocations; and

generate a test statistic for comparing the categorized combined empirical probabilities, wherein the test statistic measures a change in the overall probability due to the missing portion of the clinical trial data associated with the subject.

98. The system of embodiment 97, wherein the processing circuitry is further configured to rank the subject based on the change in the overall probability due to the missing portion of the clinical trial data associated with the subject.
99. The system of embodiment 97, wherein the processing circuitry is further configured to, for the subject, impute the missing portion of the clinical trial data associated with the subject.
100. The system of embodiment 99, wherein the processing circuitry is configured to impute the missing portion more than once at different times.
101. The system of embodiment 97, wherein the processing circuitry, when tracking which groups contain the subject, is configured to generate a mapping of the groups containing the subject in the respective allocation of the multiple allocations.
102. The system of embodiment 97, wherein the processing circuitry, when tracking which groups contain the subject, is configured to store, in a data structure, identifiers of the groups containing the subject for the multiple allocations.
103. The system of embodiment 97, wherein the processing circuitry, when generating the test statistic, is configured to apply a transformation to each of the categorized combined empirical probabilities.
104. The system of embodiment 97, wherein the processing circuitry, when generating the test statistic, is configured to determine a Hodges-Lehmann-Sen estimator.
105. The system of embodiment 60, wherein the processing circuitry is configured to generate sufficient allocations to achieve a desired precision or statistical level of significance.
106. The system of embodiment 60, wherein the processing circuitry is further configured to select one or more test statistics for comparison prior to receiving the clinical trial data.
107. The system of embodiment 60, wherein the treatment level comprises at least one of a treatment intensity, a treatment dosage, and a treatment frequency.
108. The system of embodiment 60, wherein the clinical trial data is data from a clinical trial of a potential agent or other intervention to treat a psychological condition, a psychological syndrome, a psychiatric disorder, or a central nervous system disease.
109. The system of embodiment 108, wherein the potential agent or the other intervention is for treating post-traumatic stress disorder (PTSD).
110. The system of embodiment 60, wherein the processing circuitry is further configured to evaluate results of the clinical trial to estimate parameters for a future clinical study.
111. The system of embodiment 60, wherein the processing circuitry is further configured to evaluate interim results of the clinical trial in order to re-estimate at least one of a group size parameter, a treatment intensity, or a treatment dosage in an adaptive clinical trial.
112. The system of embodiment 60, wherein the processing circuitry is further configured to evaluate safety of a treatment being assessed in the clinical trial.
113. The system of embodiment 112, wherein the processing circuitry, when evaluating the safety of the treatment, is configured to assess if a toxicity due to the treatment is observed.
114. The system of embodiment 60, wherein the clinical trial data comprise data based on one or more clinical assessment scales.
115. The system of embodiment 114, wherein the clinical trial data comprise data based on assessment domains of the one or more clinical assessment scales, and wherein each assessment domain comprises a plurality of assessment items.
116. The system of embodiment 60, wherein the clinical trial data comprise data collected from trials at one or more locations, and wherein the plurality of groups is further organized based on the one or more locations.
117. A pharmaceutical or biologic agent, a composition of one or both, a non-pharmaceutical or non-biologic treatment of a disorder, disease, or condition, or a combination of any thereof characterized in that the approval of the agent, composition, treatment, or combination for marketing at least in part, relates to or depends on an assessment of an efficacy and safety of the agent, composition, treatment, or combination based on clinical trial data from a clinical trial of the agent, composition, treatment, or combination determined using the method of one or more of embodiments 1 to 59 or the system of one or more of embodiments 60 to 116.
118. An application for marketing approval of a pharmaceutical or biologic agent, a composition of one or both, and/or a use thereof, a non-pharmaceutical or non-biologic treatment of a disorder, disease, or condition, or a combination of any of them, which application is characterized at least in part by an assessment of an efficacy and safety of the agent, composition, treatment, or combination based on clinical data from a clinical trial of the agent, composition, treatment, or combination determined, at least in part, using the method of one or more of embodiments 1 to 59 or the system of one or more of embodiments 60 to 116.
119. Selling, offering for sale, or importing a pharmaceutical or biologic agent, a composition of one or both, a non-pharmaceutical or non-biologic treatment of a disorder, disease or condition, or a combination thereof characterized in that approval of the agent, composition, treatment or combination for marketing, at least in part, relates to or depends on an assessment of an efficacy and safety of the agent, composition, treatment, or combination based on clinical trial data from a clinical trial of the agent, composition, treatment or combination determined, at least in part, using the method of one or more of embodiments 1 to 59 or the system of one or more of embodiments 60 to 116.
120. Marketing a pharmaceutical or biologic agent, composition, or combination thereof characterized in that the agent's approval for marketing was based on an application that is characterized at least in part by an assessment of an efficacy and safety of the agent, composition, or combination based on of clinical trial data from a clinical trial of the agent using the method of any one of embodiments 1 to 59 or the system of any one of embodiments 60 to 116.
121. A pharmaceutical or biologic agent, a composition of one or both, a non-pharmaceutical or non-biologic treatment of a disorder, disease, or condition or a combination of any of them characterized in that approval of the agent, composition, treatment or combination for marketing, at least in part, relates to or depends on an assessment of an efficacy and safety of the agent, composition, treatment, or combination based on data that assess one or both of the bioequivalence or lack of inferiority of the agent, composition, treatment, or combination as compared to an existing agent, composition, treatment or combination, wherein assessing the efficacy and safety of the agent, composition, treatment, or combination based on the data uses, at least in part, the method of one or more of embodiments 1 to 59 or the system of one or more of embodiments 60 to 116.
122. A method of assessing one or both of the bioequivalence or lack of inferiority of a pharmaceutical or biologic agent, a composition of one or both, and/or a use thereof, a non-pharmaceutical or non-biologic treatment of a disorder, disease, or condition, or a combination of any of them as compared to an existing agent, composition, use, treatment or combination of any of them, the method comprising the step of assessing an efficacy and safety of the agent, composition, treatment, or combination based on data related to one or both of the bioequivalence or lack of inferiority wherein assessing the efficacy and safety of the agent, composition, treatment, or combination based on the data uses, at least in part, the method of one or more of embodiments 1 to 59 or the system of one or more of embodiments 60 to 116.
123. Submission of an application for marketing approval of a pharmaceutical or biologic agent, a composition of one or both, and/or a use thereof, a non-pharmaceutical or non-biologic treatment of a disorder, disease, or condition, or a combination of any of them, the application being characterized at least in part by an assessment of the an efficacy and safety of the agent, composition, treatment, or combination based on data related to one or both of the bioequivalence or lack of inferiority of the agent, composition, treatment, or combination as compared to an existing agent, composition, treatment or combination of any of them, wherein assessing the efficacy and safety of agent, composition, treatment, or combination based on the data uses, at least in part, the method of one or more of embodiments 1 to 59 or the system of one or more of embodiments 60 to 116.
124. Selling, offering for sale, or importing a pharmaceutical or biologic agent, a composition of one or both, a non-pharmaceutical or non-biologic treatment of a disorder, disease or condition, or a combination of any of them characterized in that approval of the agent, composition, treatment of combination for marketing at least in part relates to or depends on an assessment of the efficacy and safety of the agent, composition, treatment, or combination based on data related to one or both of the bioequivalence or lack of inferiority of the agent, composition, treatment or combination as compared to an existing agent, composition, treatment or combination of any of them, wherein assessing the efficacy and safety of the agent, composition, treatment, or combination based on the data uses, at least in part, the method of one or more of embodiments 1 to 59 or the system of one or more of embodiments 60 to 116.
125. A method of assessing an efficacy and safety of an agent, composition, treatment, or combination based on one of more toxicities or adverse events or a combination thereof observed after administration to a subject of a pharmaceutical or biologic agent, or a composition of one or both, or after treating a subject for a disease, disorder or condition, with a non-pharmaceutical or non-biologic therapy or a combination of them, the method comprising the step of assessing the efficacy and safety of the agent, composition, treatment, or combination based on the one of more toxicities or adverse events using, at least in part, the method of one or more of embodiments 1 to 59 or the system of one or more of embodiments 60 to 116.

1. A method for assessing an efficacy and safety of an agent, composition, treatment, or combination based on clinical trial data, the method comprising:
receiving clinical trial data, wherein the clinical trial data comprise a data structure comprising data corresponding to subjects in the clinical trial, wherein the subjects have been organized into a plurality of groups based on treatments or treatment levels, and wherein at least one group of the plurality of groups is a control group; and
generating multiple treatment allocations of the data structure, wherein generating the multiple treatment allocations comprises, for each of the multiple treatment allocations, reorganizing, at random, the subjects along with the corresponding data to generate further pluralities of groups without regard to the respective treatment, treatment level, or status as the control group in the data structure, and wherein, for each multiple treatment allocation, a probability for a subject being reorganized into a group of the further pluralities of groups is comparable to a probability of the subject having been organized into a group of the plurality of groups based on the treatments or the treatment levels.

2. (canceled)

3. The method of claim 1, wherein each of the multiple treatment allocations is generated using a randomization protocol.

4. The method of claim 3, wherein generating the multiple treatment allocations using the randomization protocol comprises executing a group algorithm to form groups of subjects based on preselected criteria for balancing the groups and at least one of a temporal sequence.

5. (canceled)

6. The method of claim 1, wherein the clinical trial data comprise ordinal data corresponding to each of the subjects, and wherein generating the multiple treatment allocations enables addressing the ordinal data when assessing the efficacy and safety of the agent, composition, treatment, or combination based on the clinical trial data.

7. The method of claim 1, wherein the clinical trial data comprise multivariate data corresponding to each of the subjects, and wherein generating the multiple treatment allocations enables analyzing the multivariate data while minimizing information loss.

8. The method of claim 7, wherein analyzing the multivariate data while minimizing information loss comprises determining importance weights for variables of the multivariate data.

9. The method of claim 1, further comprising:
determining a statistical significance based on an overall probability and the multiple treatment allocations of the data structure, wherein:
determining the statistical significance comprises generating the overall probability based on the data structure and each of the multiple treatment allocations of the data structure; and
generating the overall probability comprises executing a combination analysis for comparing test statistics between each group of the plurality of groups of the data structure and for comparing test statistics between each group of each of the further pluralities of groups of the multiple treatment allocations.

10. The method of claim 9, wherein executing the combination analysis comprises:
determining test statistics for comparing between groups in the plurality of groups and between groups in each of the further pluralities of groups, wherein the test statistics correspond to each of the components of the data structure and the multiple treatment allocations, wherein the test statistics are medians corresponding to the components of the data structure and the multiple treatment allocations;
determining empirical probabilities for each component of the data structure and the multiple treatment allocations based on the test statistics, wherein the empirical probabilities are determined based on ranking the test statistics;
combining the empirical probabilities, wherein combining the empirical probabilities comprises applying a combining function to the empirical probabilities; and
generating the overall probability based on the combined empirical probabilities.

11. (canceled)

12. (canceled)

13. The method of claim 10, wherein a subject of the clinical trial is missing a portion of the clinical trial data associated with one or more of a subject in the clinical trial, further comprising:
tracking which groups contain the subject in the respective further plurality of groups of each treatment allocation of the multiple treatment allocations;
based on the tracking of the subject, categorizing the combined empirical probabilities in correspondence with the groups containing the subject in each of the multiple treatment allocations; and
generating a test statistic for comparing the categorized combined empirical probabilities, wherein the test statistic measures a change in the overall probability due to the missing portion of the clinical trial data associated with the subject, wherein generating the test statistic comprises applying a transformation to each of the categorized combined empirical probabilities.

14. The method of claim 13, further comprising, for the subject, imputing the missing portion of the clinical trial data associated with the subject.

15. The method of claim 13, wherein tracking which groups contain the subject comprises generating a mapping of the groups containing the subject in the respective treatment allocation of the multiple treatment allocations.

16. (canceled)

17. (canceled)

18. A system for assessing an efficacy and safety of an agent, composition, treatment, or combination based on clinical trial data, the system comprising:
one or more input/output (I/O) paths for receiving and transmitting data; and
processing circuitry coupled to the one or more I/O paths and configured to:
receive, via the one or more I/O paths, clinical trial data, wherein the clinical trial data comprise a data structure comprising data corresponding to subjects in the clinical trial, wherein the subjects have been organized into a plurality of groups based on treatments or treatment levels, and wherein at least one group of the plurality of groups is a control group; and
generate multiple allocations of the data structure;
wherein the processing circuitry, when generating the multiple allocations, is configured to reorganize, at random for each of the multiple allocations, the subjects along with the corresponding data to generate further pluralities of groups without regard to the respective treatment, treatment level, or status as the control group, and wherein, for each multiple treatment allocation, a probability for a subject being reorganized into a group of the further pluralities of groups is comparable to a probability of the subject having been organized into a group of the plurality of groups based on the treatments or the treatment levels.

19. (canceled)

20. The system of claim 18, wherein the processing circuitry is configured to generate each of the multiple allocations using a randomization protocol.

21. The system of claim 20, wherein the processing circuitry, when generating the multiple treatment allocations using the randomization protocol, is configured to execute a group algorithm to form groups of subjects based on preselected criteria for balancing the groups (and at least one of a temporal sequence).

22. (canceled)

23. The system of claim 18, wherein the clinical trial data comprise ordinal data corresponding to each of the subjects, and wherein the processing circuitry, when generating the multiple allocations, is configured to address the ordinal data when assessing the efficacy and safety of the agent, composition, treatment, or combination based on the clinical trial data.

24. The system of claim 18, wherein the clinical trial data comprise multivariate data corresponding to each of the subjects, and wherein the processing circuitry, when generating the multiple allocations, is configured to analyze the multivariate data while minimizing information loss.

25. The system of claim 24, wherein the processing circuitry, when analyzing the multivariate data while minimizing information loss, is configured to determine importance weights for variables of the multivariate data.

26. The system of claim 18, wherein the processing circuitry is further configured to determine a statistical significance based on an overall probability and the multiple allocations of the data structure, and
wherein the processing circuitry is configured to:
when determining the statistical significance, generate the overall probability based on the data structure and each of the multiple allocations of the data structure; and
when generating the overall probability, execute a combination analysis for comparing test statistics between each group of the plurality of groups of the data structure and for comparing test statistics between each group of each of the further pluralities of groups of the multiple allocations.

27. The system of claim 26, wherein:
the processing circuitry, when executing the combination analysis, is configured to:
determine test statistics for comparing between groups in the plurality of groups and between groups in each of the further pluralities of groups, wherein the test statistics correspond to each of the components of the data structure and the multiple allocations, and wherein the test statistics are medians corresponding with the components of the data structure and the multiple allocations;
determine empirical probabilities for each component of the data structure and multiple allocations based on the test statistics;
combine the empirical probabilities; and
generate the overall probability based on the combined empirical probabilities;
the processing circuitry, when combining the empirical probabilities, is configured to apply a combining function to the empirical probabilities; and
the processing circuitry is configured to determine the empirical probabilities based on ranking the test statistics.

28. (canceled)

29. (canceled)

30. The system of claim 27, wherein a subject of the clinical trial is missing a portion of the clinical trial data associated with the subject, and wherein the processing circuitry is further configured to:
track which groups contain the subject in the respective further plurality of groups of each allocation of the multiple allocations;
based on the tracking of the subject, categorize the combined empirical probabilities in correspondence with the groups containing the subject in each of the multiple allocations; and
generate a test statistic for comparing the categorized combined empirical probabilities, wherein the test statistic measures a change in the overall probability due to the missing portion of the clinical trial data associated with the subject, wherein the processing circuitry, when generating the test statistic, is configured to apply a transformation to each of the categorized combined empirical probabilities.

31. The system of claim 27, wherein the processing circuitry is further configured to, for the subject, impute the missing portion of the clinical trial data associated with the subject.

32. The system of claim 27, wherein the processing circuitry, when tracking which groups contain the subject, is configured to generate a mapping of the groups containing the subject in the respective allocation of the multiple allocations.

33. (canceled)

34. (canceled)

35. A pharmaceutical or biologic agent, a composition of one or both, a non-pharmaceutical or non-biologic treatment of a disorder, disease, or condition, or a combination of any thereof characterized in that the approval of the agent, composition, treatment, or combination for marketing at least in part, relates to or depends on an assessment of the efficacy and safety of the agent, composition, treatment, or combination based on clinical trial data from a clinical trial of the agent, composition, treatment, or combination determined using the method of claim 1.

36. A pharmaceutical or biologic agent, a composition of one or both, a non-pharmaceutical or non-biologic treatment of a disorder, disease, or condition or a combination of any of them characterized in that approval of the agent, composition, treatment or combination for marketing, at least in part, relates to or depends on an assessment of the efficacy and safety of the agent, composition, treatment, or combination based on data that assess one or both of the bioequivalence or lack of inferiority of the agent, composition, treatment, or combination as compared to an existing agent, composition, treatment or combination, wherein assessing the efficacy and safety of treatments based on the data uses, at least in part, the method of claim 1.
